(19) United States
(12) Patent Application Publication
(10) Pub. No.: US 2010/0161010 A1
(43) Pub. Date: Jun. 24, 2010

(54) Perceptible Apparatus and Methods for Reactive Effect

(76) Inventor: David A. Thomas, Longmont, CO (US)

Correspondence Address: JACKSON ESQUIRE;ROGER A. JACKSON, 209 KALAMATH STREET, UNIT 9, DENVER, CO, 80223-1348, US

(21) Appl. No.: 12/639,845

(22) Filed: Dec. 16, 2009

Related U.S. Application Data
(63) Continuation-in-part of application No. 10/596,376, filed on May 12, 2007, now abandoned.
(60) Provisional application No. 60/529,462, filed on Dec. 11, 2003.

(51) Int. Cl. A61N 5/06; A61M 21/00

(52) U.S. Cl. 607/88; 600/27

(57) ABSTRACT

A perceptible apparatus and method for creating a subtle energy field is disclosed. The apparatus includes a light source and a plurality of configuration elements that are in effectual communication with the light source, wherein the configuration elements are adjustably selectable to produce subtle energy of different vibrations. The plurality of configuration elements are preferably selected from the group consisting essentially of a spectral color analysis of the light source, a brightness of light pulses from the light source, a wave form of light pulses through a duration of light pulse(s), and a frequency of light pulse transitions from “on” to “off,” or transitions between different brightness levels. Also included in the plurality of configuration elements is an emitter in the form of a translucent element that is in light wave communication with the light source to further produce changes in the subtle energy field.

### RELATED PATENT APPLICATIONS

This continuation in part patent application claims priority from U.S. patent application Ser. No. 10/596,376 filed on May 12, 2007 by David Thomas of Longmont, Colo., Bertrand Babinet of Lyons, Colo., and Jon Tempest of Parker, Colo. that claims priority from a Patent Cooperation Treaty patent application having international application number PCT/US04/41238 filed on Dec. 10, 2004 by Star Energetics Holding Company, L.L.C. that claims priority from a U.S. provisional patent application Ser. No. 60/529,462 filed on Dec. 11, 2003 by David Thomas of Longmont, Colo., Bertrand Babinet of Lyons, Colo., and Jon Tempest of Parker, Colo.

### TECHNICAL FIELD

The present invention relates to a device that creates a subtle-energy field, similar in nature to the bio energetic field of the human body. This apparatus and associated methods have a number of applications, including holistic health/energy-balancing modalities.

### BACKGROUND OF INVENTION

This invention is applicable for many types of energetic treatment modalities. Although subtle-energy (the basis of these modalities) is not yet measurable by conventional, scientific means, its existence is widely accepted among holistic-health practitioners. Homeopathic remedies are an example of a widely used modality, which is based on subtle energy. Many practitioners and other practitioner 55 or user 54 claim varying degrees of ability to “sense” subtle energy.

In addition to subtle-energy modalities, there are other modalities that use colored lights. Often, the lights are colored with the seven colors of the rainbow, being red, orange, yellow, green, blue, indigo, and violet. In many holistic modalities, it is believed that these seven colors of the rainbow map to certain energy centers of the body, called chakras.

There are a number of devices on the market that are used in the holistic-health field. Some of them generate colored light; some of them claim to generate a subtle energy field; some work in other ways. Many of them are quite expensive, in the range of $1,000 to as much as $18,000. Typically, the more expensive devices are owned by practitioners. User 54s must schedule an appointment with their practitioner in order to use a device. Additionally, the devices on the market frequently support only one treatment modality. This fact prevents them from becoming widely used, and keeps them expensive, as not many units are made and sold. What is needed is a low-cost, flexible, effective device that practitioners can sell, rent, or otherwise provide to their user 54s for home-use. This invention presents a breakthrough in energetic treatment in that it is a low-cost technology, highly effective, user-friendly, and programmable/customizable to support a variety of treatment modalities.

Users of the exemplary embodiment of the invention have experienced relief from a variety of physical discomforts; including headaches, back aches, neck/knee/shoulder pain, carpal tunnel syndrome, nausea, fatigue, and PMS. Early experiments suggest that the invention may also have utility in resolving psychological issues, as well.

U.S. Pat. No. 5,947,908 to Morris discloses an apparatus for deriving colors that have either a positive or negative impact upon an individual. Morris uses biofeedback means to determine if a color presented to a particular individual is healthful or stress producing. Morris uses color but does not have any subtle energy producing means. U.S. Pat. No. 4,940,323 to Downing discloses a visible light based stimulator device. Downing has a brief discussion about a technology for generating photons that “have a great affinity to affect the human body by interaction with the body's own biomagnetic field”. Downing's technology is very different from that used in the present invention; however, it does present another example of a patent which references the notion of subtle energy. U.S. Pat. No. 6,016,450 to Crock presents a treatment method and apparatus that is vastly different from the present invention, but includes an excellent history of research into the human aura (biofield). U.S. Pat. No. 6,602,275 to Sullivan discloses a device that uses a plurality of flashing LEDs to produce a healing effect on an individual, but does not have a means of transmuting the light energy generated into a subtle energy field. Sullivan requires a great many LEDs to be effective. Compared to the aforementioned prior art, the present invention has the advantages of low cost, portability and substantial effectiveness.

### SUMMARY OF INVENTION

Broadly, the present invention is a perceptible apparatus for use in helping to create a reactive effect upon a user, the present invention includes control circuitry operative to generate a one of a plurality of selected signals that are preferably pulse width modulation signals or alternative signals. Also included is a means for producing a variable perceptible output in response to one of a plurality of selected pulse width modulation signals and a translucent element adjacent to the means for producing a variable perceptible output, the translucent element is operative to diffuse and emit the variable perceptible output. Note that “perceptible output” includes both light and subtle energy as described herein.

In addition a method of using a perceptible apparatus for use in helping to create a reactive effect upon a user, comprises the steps of, positioning the user, providing the perceptible apparatus that includes, control circuitry operative to generate a one of a plurality of selected signals. Also provided is a means for producing a variable perceptible output in response to one of a plurality of selected signals, and a translucent element adjacent to the means for producing a perceptible output, with the translucent element being operative to diffuse and emit the perceptible output. A further step is in locating the perceptible apparatus to a selected position adjacent to the user, continuing to a step of activating the perceptible apparatus that is operational to illuminate the perceptible output in response to one of a plurality of selected signals. Also a step is in determining the characteristics of the selected signals to produce the desired effect.

Further, a method for calibrating a perceptible apparatus for use in helping to create a reactive effect upon a user, comprising the steps of, providing the perceptible apparatus that includes, control circuitry operative to generate singularly or simultaneously in each one of a plurality of selected modes a plurality of selected signals for each the mode, a plurality of LEDs, with each one LED variably illuminated in response to the plurality of selected signals for each one of a plurality of selected modes, and a translucent element adjacent to the plurality of LEDs, the translucent element is operative to diffuse and emit one light illuminated or a plurality of lights illuminated, all being operative to generate a subtle energy field, and further including calibration circuitry for each one of the LED's that is operational to help make brightness of each one of the LED's consistent amongst the plurality of different LEDs. Another step of providing a calibration device that includes a shroud that occludes substantially all external environment light from the translucent element and calibration device circuitry that provides a signal that is displayed indicating brightness and/or color of the LEDs. A further step of adjusting brightness using the calibration circuitry and calibration device display that is operational to further help make brightness of each one of the LED's consistent amongst the plurality of different LEDs from one unit to the next. A yet further step of adjusting color by using the calibration device display by replacing a selected LED that is operational to further help make color of each one of the LED's consistent amongst the plurality of different LEDs from one unit to the next.

These and other objects of the present invention will become more readily appreciated and understood from a consideration of the following detailed description of the exemplary embodiment(s) of the present invention when taken together with the accompanying drawings, in which;

### BRIEF DESCRIPTION OF DRAWINGS

FIG. 1 is a perspective view of the exemplary embodiment of the present invention;

FIG. 2 is a perspective view of the exemplary embodiment of this invention, with the translucent element 31 removed, to show how it plugs into the translucent element slot 47 in the top of housing 33, the power plug 35 is removed, exposing the power jack 48;

FIG. 3 is a perspective view of the exemplary embodiment of the present invention with the housing 33 removed, some of the parts on the circuit board 43 are omitted for clarity, translucent element 31 is elevated in this drawing, exposing the slots 49 in the translucent element supports 41 which properly locate the translucent element 31 with respect to the LEDs 44, 45, and 46;

FIG. 4 is a side view of the exemplary embodiment of the present invention with housing 33 removed, some of the parts on the circuit board 43 are omitted for clarity, translucent element 31 is shown resting in the slots 49 in translucent element supports 41, which properly locate the translucent element 31 with respect to the LEDs 44, 45, and 46;

FIG. 5 is a side view of the translucent element support 41 showing the translucent element alignment slot 49 and circuit board mounting slot 52;

FIG. 6 is a rear view of the exemplary embodiment of the present invention with housing 33 removed, some parts of the circuit board 43 are omitted for clarity, the translucent element 31 is shown resting in the slots 49 in the translucent element supports 41, in its proper relationship to LEDs 44, 45, and 46;

FIG. 7 is a schematic diagram of the circuit used in the exemplary embodiment of the present invention;

FIG. 8 is a perspective view of the exemplary embodiment of the present invention and the calibration device 121, the calibration device 121 is in position to be inserted into the exemplary embodiment of the present invention for the purpose of calibration;

FIG. 9 is an exploded perspective view showing the major components of the exemplary embodiment of the calibration device 121;

FIG. 10 is a schematic diagram of the circuit used in the exemplary embodiment of the calibration device 121;

FIG. 11 is a flowchart depicting the main functional loop of the firmware in the exemplary embodiment of the present invention of the calibration device;

FIG. 12 is a flowchart depicting the algorithm used by the firmware in the exemplary embodiment of the calibration device to determine the brightness of the red, green, and blue channels, and thus the color and brightness of light being measured;

FIG. 13 is a flow chart depicting the main functional loop of the firmware in the exemplary embodiment of the present invention;

FIG. 14 is a flowchart depicting the algorithm used by the firmware in the exemplary embodiment of the present invention to determine duty cycles of red, green, and blue based upon selected settings;

FIG. 15 is a flowchart depicting the process the present invention uses to determine duty cycles red, green, and blue based upon the knob settings;

FIG. 16 is a flowchart depicting the algorithm or firmware of the exemplary embodiment of the present invention uses to determine the proper mode being clearing or manual;

FIG. 17 is a flowchart depicting the algorithm the exemplary embodiment of the present invention uses to calculate the duty cycles for clearing mode;

FIG. 18 is a block diagram depicting the major components of the exemplary embodiment of the present invention;

FIG. 19 is an example of a symmetrical pattern of operation used for the clearing mode in the exemplary embodiment of the invention;

FIG. 20 depicts a user 54 using an exemplary embodiment of the present invention;

FIG. 21 depicts a user 54 using an exemplary embodiment of the present invention in front of a reflective mirror 66;

FIG. 22 is a flowchart depicting the steps for using an exemplary embodiment of the present invention in manual mode;

FIG. 23 is a flowchart depicting the steps for using an exemplary embodiment of the present invention in clearing mode;

FIG. 24 is a flowchart depicting the steps for using an exemplary embodiment of the present invention in a program mode;

FIG. 25 is a flowchart depicting the steps for using the present invention to replicate a particular energetic effect;

FIG. 26 is a perspective view of the exemplary embodiment of the present invention with a container of liquid 80 in position to be encoded with a subtle energy;

FIG. 27 is a perspective view of the exemplary embodiment of the present invention with a crystal 81 in position to be energetically cleared;

FIG. 28 shows the measurements of the translucent element 31 that are used in the exemplary embodiment of the program mode example;

FIG. 29 is a flowchart showing the calculation of duty cycles for the chakra modes;

FIG. 30 is a flow chart for configuring and using the perceptible apparatus for clearing;

FIG. 31 is a flow chart for configuring and using the perceptible apparatus for creating a sacred space;

FIG. 32 is a flow chart for a method for working with the biofield of an individual;

FIG. 33 is a flow chart for a method for working with the biofield of an individual using preprogrammed settings and/or programs;

FIG. 34 is a flowchart for a scanning technique method for working with the biofield of an individual using manual settings; and

FIG. 35 is a continuation of the FIG. 34 flowchart for a scanning technique method for working with the biofield of an individual using manual settings.

### REFERENCE NUMBER IN DRAWINGS

30. Perceptible apparatus

31. Translucent element

32. Display

33. Common housing

34. Wall Transformer

35. Power Plug

36. Knob 1 (red)

37. Knob 2 (green)

38. Knob 3 (blue)

39. Knob 4 (mode-select)

40. Housing bottom

41. Translucent element support

42. Display standoffs

43. Circuit board

44. Red LED

45. Green LED

46. Blue LED

47. Translucent element slot

48. Power jack

49. Translucent element alignment slot

51. Circuit

52. Circuit board mounting slot

54. User

55. Practitioner

60. Control circuitry

62. Pulse width modulation signals

64. Means for producing a variable perceptible output

65. Diffused and emitted variable perceptible output

66. Reflective minor

68. Calibration circuitry

70. Mode select circuitry

71. LED to translucent element clearance

72. Distance between the circuit board 43 and the lower edge of the translucent element 31

80. Liquid container

81. Crystal

### REFERENCE NUMERALS USED IN CALIBRATION DEVICE

121. Calibration device

122. Power jack—calibration device

123. Display—calibration device

124. Housing—calibration device

125. Calibration device circuitry

126. Translucent element shroud—calibration device

127. Ribbon cable—calibration device

128. Color sensor chip

129. Color sensor chip slot

130. Shroud half with cutaway

131. Shroud half without cutaway

132. Cutaway for translucent element

133. Ribbon cable slot

134. External environment light

135. Periphery of translucent element 31

136. Textured surface of periphery 135

137. Annular shape of translucent element 31

138. Plurality of voids of translucent element 31

139. Planar shape of translucent element 31

### REFERENCE DESIGNATORS USED IN SCHEMATIC DIAGRAM OF PERCEPTIBLE APPARATUS

U1 Microcontroller—Microchip Technologies—PIC 18F252-I/SP

U2 LCD Display—Lumex—LCM-S01602DTR/A

U3 Clock oscillator, ½ size, 20 MHZ—Fox—H5C2E-200

U4 Quad op-amp—Microchip Technologies—MCP604-I/P-ND

U5 Voltage Regulator, 5V—On semi—MC78L05ACP

C1 0.1 uf capacitor

C2 0.1 uf capacitor

C3 100 uf electrolytic capacitor

C4 0.1 uf capacitor

Q1 Power mosfet transistor, logic level—Fairchild Semiconductor—NDP4060L

Q2 Power mosfet transistor, logic level—Fairchild Semiconductor—NDP4060L

Q3 Power mosfet transistor, logic level—Fairchild Semiconductor—NDP4060L

D1 1 amp Diode

LED1 Blue Superflux LED—Lumileds—HPWN-MB00

LED2 Green Superflux LED—Lumileds—HPWN-MG00

LED3 Red Superflux LED—Lumileds—HPWT-RD00-00000

R1 10K ¼ watt

R2 5K pot—Xicon—(Mouser part number 317-2091-5K)

R3 5K pot—Xicon—(Mouser part number 317-2091-5K)

R4 5K pot—Xicon—(Mouser part number 317-2091-5K)

R5 5K pot—Xicon—(Mouser part number 317-2091-5K)

R6 22 ohm ½ watt

R7 0 ohm resistor

R8 22 ohm ½ watt

R9 0 ohm resistor

R10 3.3K ¼ watt

R11 3.3K ¼ watt

R12 10 ohm ½ watt

R13 22 ohm ½ watt

R14 10 ohm ½ watt

R15 1K trimpot, 20 turn—Vishay—(Mouser part number 72-T93XB-1K)

R16 1K trimpot, 20 turn—Vishay—(Mouser part number 72-T93XB-1K)

R17 0 ohm resistor

R18 3.3K ¼ watt

R19 1K trimpot, 20 turn—Vishay—(Mouser part number 72-T93XB-1K)

R20 10 ohm ½ watt

R21 20K trimpot (Mouser part number 531-PT10H-20K)

R22 4.7K ¼ watt

R23 4.7K ¼ watt

R24 4.7K ¼ watt

### REFERENCE DESIGNATORS USED IN SCHEMATIC DIAGRAM OF CALIBRATION DEVICE

U10 Microcontroller—Microchip Technologies—PIC 18F252-I/SP

U20 LCD Display—Lumex—LCM-S01602DTR/A

U30 Clock oscillator, ½ size, 20 MHZ—Fox—H5C2E-200

U40 Color sensor chip—TAOS inc.—TCS230

U50 Voltage Regulator, 5V—On semi—MC78L05ACP

C10 0.1 uf capacitor

C20 0.1 uf capacitor

C30 100 uf electrolytic capacitor

C40 0.1 uf capacitor

D10 1 amp Diode

R100 10K ¼ watt

R200 20K trimpot (Mouser part number 531-PT10H-20K)

Glossary of terms;

Animal Magnetism—see “Energy”

Aura—see biofield

Baraka—see “energy”

420 Base frequency—Current embodiments of the present invention use Pulse-Width Modulation (PWM) to pulse the lights. The base frequency is the frequency of the light pulses.

Biofield—a field of chi or life-force energy in and around an organism. Also sometimes referred to as an “aura,” or “energy field.”

Body energies—see “biofield”

Body Talk—BodyTalk is a simple and effective holistic therapy that allows your body's energy systems to be re-synchronized so they can operate as nature intended. (Definition from Body Talk website.)

CAM—see “complimentary and alternative medicine”

Chi—see “energy”

Clearing—raising the vibration of a person, place, or thing. Removal of “negative” energies.

Clearing Mode—The present invention has the capability of clearing objects or spaces, and to a certain degree, organisms. This can be done through use of a “clearing mode,” which may be designed based on input from a Master for this purpose and/or programmed into the firmware of the invention. In the embodiment of the present invention currently marketed as the Perkl-Light, clearing mode is a program in which the invention sequences through 3072 different configurations in approximately 24 seconds. This works as a sort of “shotgun approach” because out of 3072 different configurations, there most likely will be one or more that is operative to help clear the particular type of negativity that one is working to clear.

Complementary and Alternative Healthcare (CAM)—a group of diverse medical and health care systems, practices, and products that are not generally considered part of conventional medicine (definition from NCCAM website)

Configuration—A “Configuration” is a set of choices regarding how particular configuration elements will be pre-set during manufacturing, for a particular embodiment of the present invention. “All examples of the present invention with a particular configuration and set on the same “setting” will produce energy of a similar vibration. (See “setting,” “configuration elements,” “user-adjustable configuration elements”)

405 Configuration elements—design/operation elements of the present invention that affect the vibration of the energy produced. These configuration elements include, but are not limited to: the base frequency of the light pulses, the wave form of the light pulses (e.g. PWM, e.g. different duty cycles), the color and brightness of the light pulses, the material the emitter is made of, the shape of the emitter, and programming (energetic definition) of the emitter, as well as physical positioning of the invention and programs (operational definition). For example, in the embodiment of the present invention currently marketed as the PERKL-LIGHT, the configuration includes a base frequency of 131 Hz, an emitter that is 4.2″ wide and made of ¼″ LEXAN, and 71 preprogrammed settings for chakras, acupuncture meridians, etc. The user-adjustable configuration element is the duty cycle of pulses of the three light sources, which are LEDs, and which are adjustable by three knobs, when the present invention is in manual mode. A user might slowly adjust one of the knobs until a desired energetic effect begins to be felt, and then repeat with the others, to determine an appropriate manual setting. Alternatively, if a user wishes to balance their chakras, they would make use of the preprogrammed settings. See “Different configurations/settings of the present invention can make a chi-like energy in different vibrations” in this description, for more details. (See “Configuration,” “User-adjustable configuration elements.”)

425 Emitter—the portion of the present invention that is operative to emit, diffuse, modify and/or amplify light and/or subtle-energy. Affects the vibration of the subtle energy emitted by the present invention.

Emotional Freedom Technique (EFT)—Emotional Freedom Techniques (EFT) is an emotional, needle free version of acupuncture that is based on new discoveries regarding the connection between your body's subtle energies, your emotions, and your health. EFT has been reported successful in thousands of cases covering a huge range of emotional, health and performance issues. (Definition from Emotional Freedom Technique website.)

Energy—an energy that is perceived by many people intuitively, but which has yet to be validated by mainstream science. This energy may be associated with an organism, a place, or a thing. In many paradigms, this energy is seen as coming from Spirit/God/Universal Energy/Source of all things. Other names for this energy include subtle energy, life-force energy, chi, ki, kundalini, Prana (yoga), Reiki Energy (Reiki), orgone, (Wilhelm Reich) baraka (Sufism), “animal magnetism” (Mesmer), odic force (Reichenbach). See “subtle energy”

Energy Block—a disruption in the flow of chi (energy) in the body and its associated energy fields. Common to many energy-healing paradigms is the notion that blockages to the flow of chi (energy) are a cause of physical, emotional or spiritual ailments/disturbances, or ailments/disturbances/imbalances in any other aspects of an individual's energy field.

Energy Clearing—see “clearing”

Energy Field—a collection of one or more energies of one or more vibrations associated with an organism, object, or a space. The biofield of an organism is an example of an energy field. The present invention also creates an energy field.

Energy Healing—Any of a variety of modalities for working with the life-force energy in the body. Examples of energy healing are Acupuncture, Reiki, Qi-Gong, Body Talk, Healing Touch and many others.

Energy Work—See “Energy Healing”

Engaging—When the energy produced by the present invention is engaging an energy block, the energy block is being changed in a useful way by the present invention. For example, the energy block may be dissolved or otherwise reduced or eliminated, facilitating balanced flow of chi. See “Energy Block.”

Enlightenment—Being able to consistently access, recognize, and express high-vibrational energies. (Inventor's definition.) Expanding your consciousness to infinite proportions, with the goal of understanding/becoming one with God/dess/Divine etc. etc.—Smonikee (definition from “about.com”)

Fifth Element—see “Energy”

Healing Touch—a relaxing, nurturing energy therapy that promotes health and well-being. Gentle touch assists in balancing your physical, mental, emotional, and spiritual well-being. Healing Touch works with your energy field to support your natural ability to heal. It is safe for all ages and works in harmony with standard medical care. (From the Healing Touch website).

High vibration—Describes energy that has the tendency to bring out emotions and expressions in an individual that are commonly considered “good.” Examples include courage, generosity, love, abundance, or growth towards enlightenment. In many energy paradigms, energies that are of a higher-vibrational nature are believed to come from Spirit/God/Source/etc.

Ki—see “Energy”

Kundalini—see “Energy”

Light-based chi generator—a device that generates an “energy” field similar to life-force (see “Energy”) using light. The present invention is an example of structure and methods relating to a light-based chi generator.

Low vibration—Describes energy that has the tendency to bring out emotions and expressions in an individual that are commonly considered “bad.” Examples include Fear, greed, or anger, or the slowing of growth towards enlightenment.

Manual Mode—a mode in the present invention in which a user can access manual settings.

Manual Mode Settings—see “Manual Settings”

Manual Settings—Settings on the present invention, using user-adjustable configuration elements, that are determined by the user to create energy of a particular vibration for a particular purpose. For example, a user may use intuitive or experiential means to determine a setting to clear a particular energy block that they are experiencing. Manual settings are in contrast to “preprogrammed settings,” which are predetermined at the factory for the convenience of the user.

Master—one who is recognized by others as having useful abilities in working with energy. The master may or may not have achieved enlightenment.

Modality—a paradigm combined with a (treatment) protocol. In the context of this description, energy-healing modalities combine a paradigm, which defines what is true and what is important, with a treatment protocol for diagnosing and/or correcting situations determined to need treatment. Examples of energy-healing modalities include Acupuncture, Reiki, chakra balancing, Qi-gong, Healing Touch, Body Talk, Emotional Freedom Technique, and many others.

NAET—Nambudripad's Allergy Elimination Techniques, also known as NAET®, are a non-invasive, drug free, natural solution to eliminate allergies of all types and intensities using a blend of selective energy balancing, testing and treatment procedures from acupuncture/acupressure, allopathy, chiropractic, nutritional, and kinesiological disciplines of medicine. (Definition from NAET website.)

NCCAM—National Center for Complementary and Alternative Medicine—a branch of the National Institute of Health. The Federal Government's lead agency for scientific research on the diverse medical and health care systems, practices, and products that are not generally considered part of conventional medicine. NCCAM explores complementary and alternative healing practices in the context of rigorous science, trains researchers, and provides authoritative information to the public and professionals

Negative Energy—energy that is of a low vibration.

Non-planar emitter—an emitter that is other than planar in its physical shape. (See emitter definition.)

Odic force—see “Energy”

Optimal Configuration—A configuration of the configuration elements of the present invention that yields energy of a high vibration.

Optimal Setting—a setting of the user-adjustable configuration elements on the present invention that produces energy of a vibration that is effective for an intended purpose.

Orgone—see “Energy”

Paradigm—a body of information about a particular subject that defines what is true and what is not, and what is important, and what is not.

139 Planar—physically shaped so as to approximate being bound by a geometric plane. For example, an emitter in which a 2-dimensional design is cut out of a sheet of ¼″ polycarbonate would be planar.

Positive Energy—subtle energy or chi that is of a high vibration.

Prana—see “Energy”

Preprogrammed settings—settings (adjustment-choices regarding configuration elements) on the present invention that have been pre-determined at the factory and made available to the user for convenience. For example, they may be programmed into the software of the invention. For example, in the embodiment of the present invention currently marketed as the PERKL-LIGHT, 71 preprogrammed settings are available for chakras, acupuncture meridians, psycho-spiritual qualities such as clarity, freedom, and joy, and many others. As such, it is very easy for the user to set the PERKL-LIGHT on the preprogrammed setting for a particular chakra to engage and balance it. Note that in the PERKL-LIGHT, the preprogrammed settings all have corresponding manual settings. This invention is also capable of embodiments where no manual settings are available, or where preprogrammed settings are made available for which there is no corresponding manual mode setting. An example of this would be an embodiment of the present invention in which the base frequency of the manual settings was 131 Hz, and this embodiment had preprogrammed settings with different base frequencies than this. In this instance, the preprogrammed setting would not have a corresponding manual mode setting on this embodiment of the present invention.

Present invention—The use of the term “present invention” includes subject matter disclosed in prior priority applications and in this continuation in part application.

Program (operational definition)—a sequence of configurations and/or settings intended to achieve a desired result. For example, the embodiment of the present invention currently marketed as the PERKL-LIGHT has a program for balancing chakras. This program sequences through a series of settings, one for each chakra. As another example, in the embodiment of the present invention currently marketed as the Perkl-Light, clearing mode is a program in which the invention sequences through 3072 different configurations in approximately 24 seconds.

Program (energetic definition)—to encode “energy” into a place or object. After programming, the place or object will have a vibration that was programmed into it. Programming is accomplished by directing “energy” towards/into the place or object. This may be accomplished by a person who is skilled at directing energy. It may also be accomplished by directing the present invention toward the place or object while it is in operation.

415 Pulse width modulation (PWM)—A practice in electrical engineering, normally for the purpose of varying the current through a load. Pulses of electricity of varying width are provided to the load. The frequency of the pulses is generally fixed. The current is varied by varying the width (duty cycle) of the pulses. Pulses of longer duration increase the duty-cycle, which in turn, increases the current through the load. The present invention uses PWM as a means of creating energies of different vibrations. Different pulse widths (duty-cycles) applied to the light sources produce energies of different vibrations.

Reiki—A Japanese word representing Universal Life Energy. The practice of Reiki is based on the belief that when spiritual energy is channeled through a reiki practitioner, the patient's spirit is healed, which in turn heals the physical body. (Adapted from a definition provided on MedicineNet.com.)

Sacred Space—an area (e.g. room, area within a room or outdoor area) that has a high vibration. (See “Vibration.”)

Setting—A user may adjust “user-adjustable configuration elements” to a particular “setting” to produce energy of a desired vibration. In the embodiment of the present invention currently marketed as the PERKL-LIGHT, “manual settings” are available in “manual mode,” in which a user adjusts the duty cycle of each light source by rotating a knob, to achieve energy of a desired vibration. “Preprogrammed settings” are available in this embodiment of the present invention to generate energy of an appropriate vibration for chakras, acupuncture meridians and the like.

400 Subtle energy—Several scientists in the United States (Tiller, Bearden, Rein, Putoff, Green, and Srinivasan) have studied Subtle Energy (SE) and its effects. Though each has developed his own nuanced theory of SE, in general they all tend to concur that SE phenomena is related to a type of unified energy, and is not just a physical field of very low magnitude. Contemporary quantum physics has mathematically described and predicted the presence of a unified energy which underlies conventional transverse electromagnetic (EM) vectors. The concept of a subtle energy underlying EM fields was first introduced by Bohm and Aharonov in describing quantum potentials as an implicate order “embedded in” our normal 3-D space. It has recently been proposed that an additional implicate order is embedded within the quantum potentials. (Definition from “Excerpt from A Subtle Energy Technology for Noise Reduction in Physical and Psychophysical Systems”—Jan, 1999 by T. M. Srinivasan, Ph.D.—QLink Co-Founder) See “Energy”

Testing Modality—a method such as muscle-testing (applied kinesiology), pendulum, or the blink technique, for discerning information. For example, testing modalities may be used to determine an appropriate setting on the present invention, or for other information relating to one's health and possible methods of treatment.

Truth—that which, if believed, helps direct one in producing desired results.

User-adjustable configuration elements—Some configuration elements are preset during manufacturing, and some are adjustable by a user after the present invention has been manufactured. “User-adjustable configuration elements” are configuration elements that are adjustable by a user. User-adjustable configuration elements are adjusted by a user to a particular “setting” to produce energy of a desired vibration. For example, in the embodiment of the present invention currently marketed as the PERKL-LIGHT, the duration (duty cycle) of the light pulses is adjustable by rotating the adjustment knobs, to produce “settings,” each having different vibrations of energy. Embodiments of the present invention that allow user-adjustment of other configuration elements than the duty cycle should be considered to be within the scope of this invention. For example, the base frequency is totally controlled by the software and need not be preset at the factory, and could therefore, be “user-adjustable configuration element” in future embodiments. User-adjustability of other configuration elements should be considered within the scope of this invention.

410 Vibration—an attribute of energy that determines its subjective qualities. For example, people, places, or things each have a “vibration.” A person who is sensitive may have the ability to recognize a person, place, or thing by its vibration. The vibration of a person, place, or thing is not static and may be changed by a variety of processes, including “clearing” or “programming,” or the application of energies from the present invention. Additionally, a vibration may be “high” (see “Positive Energy”) or “low” (see “Negative Energy”) and may be raised or lowered by a variety of processes

### DETAILED DESCRIPTION

In general the present invention deals with the generation of a subtle-energy field. This subtle energy field may be very similar to the life-force in a person's body, an animal, or a plant.

### Arguments for the Existence of Chi

Similar Information about Chi was Determined Independently by Different Cultures and Individuals

Throughout history, the maintenance of health has been a human endeavor of the highest priority. Prior to advent of medical science, there were numerous healing modalities that flourished, many of which are still in use today. Particularly, Yoga, Qi Gong, Acupuncture, Reiki, and various other forms of biofield/subtle-energy therapies (a.k.a. energy healing) have been used for hundreds, and in some cases, thousands of years.

Central to all of these modalities is the notion of a life-force energy that is involved in nearly every aspect of physical, emotional, mental, and spiritual health. There are many names for this energy, “Chi,” “Ki,” in China and Japan, “kundalini” in India. Yogic traditions refer it as “Prana.” Sufism teaches about an essence of life called “baraka.” Aristotle believed in a “fifth element.” Mesmer studied an elusive force he called “animal magnetism.” Baron Karl Von Reichenbach (the inventor of Kerosene) studied a version of this energy, which he called the “odic force.” Wilhelm Reich studied a similar energy which he called “orgone.”

More interestingly, there is a high degree of similarity between the energies described in each of these paradigms. Common among many of them are the following attributes:

The notion of a “life-force” energy that is universal—that is in all things, and which flows from a Creator (i.e. God, Spirit, Universal Mind, Source Energy, etc.)

The notion that there are energy systems that regulate how life-force energy flows in the body. Chakras, acupuncture meridians, and doshas (Ayurvedic) are three examples.

The notion that “balance” in this energy is a desirable state that contributes to a state of optimal health, and that “energy blocks,” can disrupt the flow of this energy, and are at the root of many physical and emotional health problems.

The notion that energy blocks can be the result of physical or emotional trauma to the individual, and can result in issues in the physical health years later

The notion that this energy is associated not just with humans and other living organisms, but also with physical spaces and objects as well

The notion that this energy can be of a higher vibrational (positive energy) or lower vibrational (negative energy) nature

The notion that energy of a lower vibrational nature can be “cleared” through a variety of techniques and raised to a higher vibration

The notion that energy can have vibrational qualities above and beyond being “high” or “low.” For example, energy can be “yin” or “yang,” “3^{rd} chakra energy,” or “fear” energy. Energy can have a “mental” or a “spiritual” quality to it, or many others.

It seems highly unlikely that so many individuals and cultures would have independently come up with such similar paradigms relating to a life-force energy if it did not, in fact, exist. The people who developed these paradigms lived thousands of miles apart, on different continents, and there were thousands of years between the development of the earliest and the most recent of these paradigms.

A Paradigm that Includes Chi can Explain Anomalous Phenomena

One of the more dramatic and well-known examples of working with chi or life-force energy is martial arts. Central to many martial arts practices is the cultivation and use of energy to support one in one's self defense. The fact that a person can break a board or a brick with their hand is anomalous within mainstream science. There is no explanation for how a person can accomplish such a feat at all, much less using the soft tissues of the hand, and without sustaining injury. It is the opinion of this inventor that the martial arts practitioner is directing their life-force energy through that brick or board, and that their hand is just the vehicle for directing this energy. It is the person's chi that breaks the board.

Modalities Involving Chi are used by Millions of Americans and being Actively Researched by the National Institute of Health

This notion of a life-force energy has been conspicuously absent from our modern, science-based healthcare paradigm. However, that is changing. The National Center for Complimentary and Alternative Medicine (NCCAM), a branch of the National Institute of Health, has been actively studying Reiki and other forms of energy healing. NCCAM describes itself as follows: “NCCAM is the Federal Government's lead agency for scientific research on the diverse medical and health care systems, practices, and products that are not generally considered part of conventional medicine. NCCAM explores complementary and alternative healing practices in the context of rigorous science, trains researchers, and provides authoritative information to the public and professionals.” Here are some excerpts from their website:

“According to the 2007 National Health Interview Survey, which included a comprehensive survey of Complementary and Alternative Healthcare (CAM) use by Americans, more than 1.2 million adults had used an energy healing therapy, such as Reiki, in the previous year. The survey also found that approximately 161,000 children had used an energy healing therapy in the previous year.”

“People use Reiki for relaxation, stress reduction, and symptom relief, in efforts to improve overall health and well-being. Reiki has been used by people with anxiety, chronic pain, HIV/AIDS, and other health conditions, as well as by people recovering from surgery or experiencing side effects from cancer treatments. Reiki has also been given to people who are dying (and to their families and caregivers) to help impart a sense of peace.”There are a number of studies ongoing and completed regarding energy healing. Current information may be obtained at the NCCAM website.

There is a YouTube Video Demonstrating a Simple Exercise that Enables most People to Feel their Own Chi as a Direct Experience

If one is curious and desires to have a first-hand experience of their own life-force energy, there is a simple exercise presented in a YouTube video. In this video, entitled “Chi Exists, here's proof (Qi),” Joseph Geyer demonstrates a simple exercise that enables a person to feel their own chi. In this exercise, he holds out his left hand, fingers aiming forward, palm facing to the right. His right hand has the first and middle fingers extended. He then positions his right hand so that the tips of the first and middle fingers are about ½″ away from the palm of the left hand, directed toward it at a 90 degree angle to the plane of the palm of the left hand. He then moves the right hand in a circular motion so as to “draw” a circle on the palm of the left hand. This is done while maintaining the approximately ½″ distance between the fingers of the right hand and the palm of the left hand, so that the hands do not touch one another. The circle is “drawn” a number of times, until sensations are felt in the palm of the left hand.

It is common for people who try the exercise presented in this video to report a variety of sensations in their left hand, for which there is no scientific explanation. Interestingly, people who try this exercise often feel sensations similar to what they feel when they position their hands over the present invention, when in operation. (See “The present invention makes an energy field similar to chi” later in this description.)

Modalities Involving Chi are Beginning to be used in Hospitals

There is even more evidence of therapies and practices involving the life-force energy of the body coming into the mainstream. According to betterhealthandliving.com, over 50 hospitals employ some form of energy healing as an adjunct to their traditional medical treatments (as of 2006).

### There are Problems with how Science is Currently Practiced Regarding Determining the Existence of Chi

Fallacy that Everything Important is Already Known

Skeptics frequently argue that there is currently no way of measuring chi—the only evidence for its existence is the experiential report of (millions of) people. The fallacy in the skeptics argument is an assumption that science already “knows” everything that is true or useful. People who really understand science know that it is a tool for advancing our understanding of the natural world, that it is an ever-expanding paradigm, and that part of this paradigm is that every discovery just leads to more questions asked—more research to be done. Nowhere in the scientific method is there any assertion of the completeness of that which has been discovered.

Science does not Presently Understand the Nature of Love, or of any Sort of Experience

And, one could argue that science is relatively useless in terms of one of the most important aspects of human experience—that of love. Yes—science has isolated some mechanisms in the brain involving endorphins and such that are involved with our experience of love. And science can talk about how love is important because it causes parents, for example, to take care of their children, furthering the survival of the species. But science has little of use to say about why love is important to us as an experience. (Note that in many energy paradigms, love is considered to be one of the highest vibrational energies there is.)

Indeed, science has little to say about the nature of experience in general. Yes—science can talk about experience in terms of neurological mechanisms that correlate with pain, pleasure, and such. But science has very little to say about who is in there having that experience of pleasure or pain. Theoretically, you could model the neurological mechanisms involved with various experiences on a computer. But science, so far, has no ability to distinguish between these mechanisms running in a person's neurological systems (in which case, someone is having an experience), and in a computer (in which case, no-one is having an experience). Science has yet to truly understand the nature of experience.

A further point about this is that science is a predominantly left-brained activity—rational, linear, and analytical. Perception of chi, in the many disciplines described herein so far, is a predominantly right-brained (intuitive) activity. In our science-based Western culture, people with the greatest analytical capabilities, and who are trained and practicing as scientists, are seen as the most reliable source of truth. And they produce many facts that are of great utility if one needs to accomplish an engineering task such as making a car or a cell phone. And yet they have little or nothing to say about the meaning of life or why we are here or what our purpose is, which are arguably among the most important areas in which to determine truth.

Evolutionary Processes are Another Source of Truth, Beyond Science

There is another source of truth that is highly overlooked in our culture, and that is the wisdom inherent in the process of evolution. Evolution is fundamentally, a vast experiment in terms of what works and what does not. If a trait or structure in an organism helps it to survive, the organism is more likely to reproduce, and the trait or structure is passed on to their progeny. Traits/structures that do not assist an organism in survival result in an organism that is less likely to reproduce, in which case the traits/structures do not get passed on to progeny, and find their way out of the gene pool. The result is something like what we see today in the natural world all around us—a myriad of organisms, each highly adapted and specialized to the world in which it lives, with traits/structures that have been tested and tested and tested, by the process of evolution. The process of evolution has determined with near-absolute certainty, the usefulness of the traits/structures involved with that organism's survival. Therefore, the process of evolution should be considered a highly effective means of determining truth—particularly in the area validating the usefulness of traits/structures of organisms.

This being the case, one could argue that any complex structure or trait that appears in a highly-evolved organism such as a human being must have applicability in terms of survival. It is clear that complex structures such as our intuitive right-brains would not exist if they did not have some ability to enhance our survival, i.e. some ability to discern truth. And yet, the types of truth that are discerned by the right brain are vastly different from those discerned by the left-brain. As such, one could argue that a widely institutionalized method for determining truth, (scientific method) that only involves left-brained processes of linear analysis, will produce incomplete truths, at best. It is the opinion of this inventor that this argument accurately describes the current relationship between mainstream science and studies of life-force energies (by whatever name), that are easily perceived by the intuition, but which the analytical mind is essentially blind to.

Our Perception of Truth is Limited because Intuitive People are Generally not Well-Respected in Our Culture

One further argument relating to this is that people tend to fall on a spectrum in terms of being more comfortable with the right-brained, intuitive, body-aware types of perceptions, vs. being more comfortable with rational, analytical processes of the left brain. Through many years in the business, the inventor has had an opportunity to talk to many people at all levels of ability at sensing and working with subtle energy. There are people who are extremely gifted intuitively as energy healers, but who have “12:00” flashing on their VCR because they could not figure out how to set the clock. At the other end of the spectrum is the stereotype of the “absent-minded professor,” that can do calculus in his head, but can't manage to put on socks that match in the morning. Given that our whole Western, scientific approach to determining “truth” revolves around the latter type of person, and that the former type of person is not generally well-respected, it is easy to see that we could end up with incomplete truths, at best.

### Review

To summarize my arguments:

Many individuals and cultures have postulated the existence of “chi” or “life-force energy—developed independently, and over thousands of years.

There is a great deal of similarity between what these individuals/cultures say about life-force energy.

It is highly unlikely that such similar information could be developed independently unless there actually was some sort of real phenomenon that was being observed.

Science does not yet know everything—there is a great deal of the natural world about which the science is not yet understood.

Science, being primarily a left-brained analytical process, does not take significant advantage of the intuitive gifts of the right brain.

The intuitive gifts of the right brain have been validated independently of science, through the process of evolution, as useful for determining at least certain types of truth related to survival.

Given that science and the scientific method make only limited use of our right-brained intuitive capabilities (which have been validated by evolution in terms of their usefulness), science/scientific method can produce only incomplete truths, at best.

It is easy to see now, that there is a huge blind-spot in our scientific paradigm, when it comes to things that are best perceived and worked with intuitively. This blind spot is big enough that a phenomenon such as chi/life-force/subtle energy, that are easily perceived by people who have highly developed intuition, could exist and be taught by many disciplines developed independently in many different cultures, and science (as currently practiced) would not be able to even be certain of its existence.

Given the above, a new criterion needs to be established regarding subtle-energy studies. Given that science, as it is currently practiced in the mainstream, does not have the tools with which to develop a useful theoretical model for subtle-energy/life-force, the role of science and the scientific method should be relegated to the answering of one simple question—“is it useful?”

### Introduction to Chi (Energy)

In the remaining portions of this description, I will refer to these energies by their different names, depending on the context. It should be understood that the names, “chi,” “ki,” “life-force energy,” “energy,” and “subtle-energy,” and “Reiki energy,” all mean essentially the same thing.

As stated earlier, there are many cultures/individuals that have independently come up with paradigms about this energy. While there are some differences in the information in these different paradigms, there is also a high degree of similarity. It can be argued that if there is an absolute “truth” about the nature of these energies, this truth could be approximated by looking at what is common among all these independently developed paradigms. A characterization of this is presented in the following sections. This characterization has, to a certain degree, been validated by our experience successfully marketing the present invention to customers operating in many different energy-healing paradigms.

### Chi comes in Different “Vibrations”

Common to many energy-healing paradigms is the notion that some energies have a different “vibration” than others. The vibration of the energy reflects subjective qualities. For example, an energy could be very “mental,” or “nurturing,” or “disturbing.” Another example is that each of the chakras has a different “vibration.” The same would be true of Acupuncture meridians. To one who is sensitive, these energies “feel” different from one-another. Additionally, energies associated with spaces such as a room or an outdoor area can also have their own vibration or energetic “feel.” Furthermore, energies associated with a person or an object can have a distinctive “feel.” For example, “That dress has an energy that reminds me of my grandmother.”

In addition to the subjective qualities described above, some energies are seen as having a “higher vibration” than others. Energies that are of a higher-vibrational nature are believed to come from Spirit/God/etc. Exposure to high-vibrational energies tends to bring out the best in people. For example, courage, generosity, love, abundance, or growth towards enlightenment. Energies that are of a “lower vibration” tend to bring out the worst in people. For example, fear, greed, or anger, or the slowing of growth towards enlightenment.

### There are Systems that Regulate how Chi Flows through the Body

Common to many paradigms relating to the life-force energy is the existence of systems that regulate how the energy flows through the body, and moreover, the consciousness of an individual. Acupuncture, for example, organizes the flow of chi into acupuncture meridians and acupuncture points. Other modalities organize the energy into chakras. Ayurvedic medicine organizes the body energies into 3 “doshas,” being “Vata,” “Pitta,” and “Kapha.” Modem techniques such as Body Talk, Healing Touch, Emotional Freedom Technique or NAET have other systems for understanding and organizing the flow of energy through the body, and for clearing imbalances in this energy.

On the surface, there is apparent disagreement between these systems. In some systems, for example, the energy is organized into chakras. In others, it is organized into acupuncture meridians. It may seem that these two, very different views on the body energy could not both be right. To further add to the confusion, there is not even agreement about how many chakras or acupuncture meridians there are. While this is generally not a cause for significant disagreement among energy healers, to a novice, it may appear that there is disagreement between these various paradigms.

This matter is resolved easily when one considers the nature of light, as it is understood by physics. In certain situations, it works better to think of light as being organized into packets of energy called “photons.” In others, it works better to think of light being a wave of electromagnetic radiation at a certain frequency. It is difficult to imagine how light can be both a particle and a wave at the same time. So clearly, light is something more than just a particle or a wave, such that sometimes it is best understood by thinking of it as a particle, and at other times, it is best understood by thinking of it as a wave.

Analogously, chi is best understood in some situations by thinking of it as organized into systems of chakras. In other situations, it is best understood by thinking of it as being organized into acupuncture meridians, doshas, etc. Similarly to light, the existence of apparently conflicting paradigms about the nature of this energy does not mean that any of these paradigms is “right” and the others are “wrong.” And it is the experience of this inventor, after talking to hundreds of energy practitioners in all different disciplines, that useful results may be obtained in any of them, depending upon the skill of the practitioner.

### There can be “Energy Blocks,” which Disrupt the Flow of Chi, Frequently Resulting in Health Problems

In most of the energy paradigms (with the possible exception of the martial arts), blockages to the flow of chi are seen as a precursor to disturbances in one's physical, mental, or emotional health. Acupuncture is one of the oldest modalities using this premise. There are many other newer ones, including Healing Touch, Body Talk, or Emotional Freedom Technique (EFT). The energy blocks can affect virtually any part of the body and can also affect the emotions and one's sense of well-being. In these energy paradigms, the clearing of energy blocks enhances the natural flow of chi through the body, and strengthens the body's natural ability to deal with stressors of many sorts.

In some cases, the energy blocks can have both a physical and an emotional component. It is commonly reported that when an energy healer starts out working to restore the flow of chi in a physical part of the body (an injured foot, for example), an emotional issue may surface. The clearing of the emotional issue through meditation or other practices can complete the healing process.

### There are “Masters” who are Highly Skilled at Working with Chi

The cultivation of the many energy-related disciplines worldwide has followed a process similar to that of the evolution of species in the natural world. In each case, the useful practices (energy disciplines) or traits/structures (organisms) have tended to be continued, while the less useful ones have fallen away. While the scientific method is highly valuable as a means of determining certain types of analytical knowledge, evolutionary processes of trial and error over long periods of time are valuable as a means of determining pragmatic truths with an element of wisdom. When an energy discipline such as yoga, Qi gong, acupuncture, or martial arts is practiced over time, this wisdom, born of thousands of years of trial and error will tend to produce a highly effective practice. In the case of yogis, it has been well documented that some of them have unusual abilities such as controlling their heart rate or various other bodily functions. In the case of martial arts practitioners, the ability to break a board or a brick could be seen as a fruit of this evolutionary process of trial and error over large amounts of time.

The main purpose of this argument is to illustrate the existence of masters—people who are highly skilled in the practice of various energy disciplines. For the purposes of this discussion, we can consider a master to be a person who is recognized by others as having useful abilities in working with energy. The master may have highly-evolved gifts and skills of perceiving and working with life-force energy fields. Like a discipline such as art or music, putting a gifted person under the tutelage of a master can yield another master. Like a discipline such as art or music, the resulting master stands on the shoulders of those before him/her, taking advantage of the wisdom that has been gained over the passage of time, and adding their own innovations/improvements to the discipline, which are then further subjected to the test of time.

### The Present Invention makes an Energy Field Similar to Chi

The present invention has been on the market, under the trade name “PERKL-LIGHT.” We consistently hear from our customers skilled in other energetic arts that the energy emitted by the present invention feels like the same energy that they are used to working with in their energy-healing practices. For example, it is very common for people using the present invention to report that the energy “feels just like Reiki energy.” It is also common for people who have no energy work experience to feel sensations such as warmth, tingling, pulsations, vibrations, coolness (as though there were a fan inside blowing air at their hand), or a sense of pressure gently pushing their hand away from the present invention. Once again, there is no scientific explanation for why people would feel these sensations. In spite of this, the vast majority of people tested have felt sensations when tested.

To a skeptic, a question remains as to whether the device is emitting anything other than visible light. To answer this, a small study was conducted on Aug. 1, 2008, at the Unique Prints Pediatric Therapy Clinic Inc., in Denver, Colo. The purpose of the study was to determine if participants could tell the difference between a real PERKL-LIGHT and a sham PERKL-LIGHT, just by feeling the energy. The sham PERKL-LIGHT was similar to the real one, except that the emitter was of black Delrin, rather than clear polycarbonate, as it is in a real PERKL-LIGHT. The sham PERKL-LIGHT also had all of its electronics disabled. The subjects were blindfolded, so they could not see the PERKL-LIGHTs. The study was supervised by Dean Radin, who is the chief scientist at the Noetic Sciences Institute. A total of 20 people participated. The results of the study were that 60.5% of the guesses were correct, which given our sample size is associated with p=0.005 or odds against chance of 200 to 1. This study was videotaped.

While a small study like this admittedly is suggestive, but not conclusive, there is one “study” that does have a statistically significant sample size (1300 customers, worldwide), that does indicate the usefulness of our technology. That is that the PERKL-LIGHT retails for just under $500, we have a 30-day return policy, and we get approximately 5% back. So approximately 19 out of 20 people are sufficiently convinced of the usefulness of our product that they choose to keep it when they could just as easily get their money back. It is highly unlikely that a product at this price point would have such a low return rate, unless it was actually providing a useful benefit for our customers.

### Different Configurations/Settings of the Present Invention can make a Chi-Like Energy in Different Vibrations

Through experimentation, it has been determined that a variety of factors contribute to the vibration of the energy produced by the present invention. In particular, the following aspects of the construction of the present invention, which we call “configuration elements,” have been determined to have a significant influence on the vibration of the subtle energy field produced:

The base frequency of the light pulses. Current embodiments of the present invention use Pulse-Width Modulation (PWM) to pulse the lights. The base frequency is the frequency of the light pulses.

The duration (i.e. duty cycle) of the light pulses

The wave form of the light pulses (e.g. Pulse Width Modulation)

Whether the light pulses are in a wave form based on a square-wave or some other shape of wave

The pattern of light pulses from a plurality of different light sources—i.e. how long is one on/off, before another one turns on/off? Or in the case of wave forms that are not based on square waves, the pattern of transitions from one illumination level to another among a plurality of different light sources?

The spectral analysis (color) of the light sources

The brightness of the light pulses

Whether the light pulses transition from “on” to “off,” or transition between different brightness levels

In the instance of a plurality of light sources of different colors, the pattern and timing of light pulses of different colors resulting from the mixing colors produced by light-sources of different colors being simultaneously illuminated. For example, if a red light source is on for 20 msec and a blue one is on for 30 msec (assuming that they both start at the same time) you get a period where the red and blue are both on for 20 msec, mixing to form purple, followed by a period where only the blue is on for 10 msec.

The material the emitter is made of (e.g. LEXAN, MAKROLON, and quartz.)

The shape of the emitter, including designs etched or otherwise put on it and any holes in it

“Programming” (energetic definition) of the emitter, by a master, by directing energy towards it

Physical positioning of the present invention in a space and/or relative to a user and/or object being “cleared,” and/or relative to other examples of the present invention including motion of the present invention relative to a user and/or relative to an object being cleared, and/or relative to other examples of the present invention

Programs (operational definition) in which one or more of the other configuration elements changes with time

There likely will be other aspects of the configuration determined at a later date, upon further research.

In terms of the design and operation of the present invention, certain configuration elements are generally preset when the invention is manufactured, while others may be adjusted by the user. A “configuration” is a set of choices regarding configuration elements that are preset when the invention is manufactured. A “setting” is a collection of choices regarding user-adjustable configuration elements that is determined by the user and to which the present invention is set, during operation. The combination of the “configuration” and the “setting” determines the vibration of the energy produced by the invention.

In our experience with this technology to date, it has been noted that some configurations and/or settings seem to affect the emotions more, others the physical body, others, different aspects of the body's energy field. It has also been noted that the energy produced by the different configurations and/or settings feels different.

It can be seen that there is virtually an infinite number of configurations/settings, given all the parameters described above. Pulsing LEDs or other light sources using a wave-form such as PWM is certainly widely-practiced. We have observed that there are particular optimal configurations and/or settings in this vast ocean of possible configurations that create this effect of a chi-like energy with a high vibration.

Given the vastness of the number of configurations of these parameters, it is not possible or practical to find these optimal configurations and/or settings by a purely rational, analytical process. A purely analytical approach is not sufficient to search an infinite problem space via brute-force. Instead, a balanced approach, involving both the rational and the intuitive is required. The left-brain is involved, because there is engineering required for the device to be designed, tested, and manufactured. Intuition is also required, because while the problem space to be explored is infinite, the optimal configurations/settings can be felt or otherwise determined intuitively.

Examples of using the mind and intuition in balance, to engineer an embodiment of the present invention with a high vibration include:

1. Using one's intuitive guidance, dreams, etc. to determine optimal choices for the configuration elements or

2. Adjusting the apparatus through a range of possible configurations and/or settings and stop when a local maximum in the desired energy characteristic is observed e.g. intuitively, or with biofeedback means. Then adjust another parameter, repeating the process in an iterative fashion until the desired energy characteristic has been optimized.

3. Using a combination of the above two approaches.

### The Present Invention can be used to Replicate the Gifts of a Master

Throughout history, people have spent great sums of money and traveled great distances to be in the presence of a master. It is widely recognized that being in the presence and “energy” of a master has certain benefits above and beyond just reading one of the master's works, for example. Physical logistics can impose some practical limitations on the number of people a master can share their energy with in a close, intimate way.

Given that intuition is such a fundamental part of the process of making the present invention, the quality of the invention is largely conditional on the intuitive gifts of the people determining the configuration and/or settings of the device. It stands to reason then that if a master is involved with the configuration and/or settings of the device, the energy produced will be of a higher vibration or quality. One can assert then, that the device is now serving as a platform for replicating the intuitive gifts of this particular master, as all similar devices manufactured in the configuration and/or settings determined by the master will produce a similar energy, that the master has deemed to be of high vibration or quality. Anyone in possession of one of these devices will have access to the energy it produces at any time.

Simply put, since the present invention is intuitively configurable, it can be used to replicate energies that have been determined to be of a high vibration or otherwise useful by a master. Additionally, the present invention may be used to provide an experience of these energies to many more people than the master would be able to see in person.

To recap, here are the steps

1. Have the master work closely with the engineers to determine an optimal configuration of the configuration elements of the present invention (example configuration elements are enumerated in the section “Different configurations/settings of the present invention can make a chi-like energy in different vibrations.” These configuration elements include the frequency, duration, wave-form, color, and brightness of the light pulses, the physical configuration of the device including especially, the emitter, “programming” (energetic definition) of the emitter, physical positioning of the invention, and the presence of programs (operational definition) that sequence through configuration elements over time.) The master will be using his intuitive abilities in working with energy to determine an optimal configuration for each of the design parameters.

2. Optionally, the master may determine specific settings on the embodiments of the present invention configured in step 1, to produce energies with vibrations for specific purposes.

3. Produce a plurality of examples of the present invention, each with the configuration determined in step 1.

4. Make the examples of the present invention with the configuration determined in step 1 available to those users who would like to experience the energy they produce.

5. The users would then power up the examples of the present invention and spend time in proximity to it, in order to experience the energies that the master deemed helpful.

6. Optionally, if the master has determined particular settings on the examples of the present invention configured as in step 1, the users may then set their examples of the present invention on a setting determined by the master, to experience the energy the master deemed useful in step 2.

7. Alternatively, some users may find settings of their own, which would cause their example of the present invention configured as in step 1 to produce energy with vibrations that are particular to their needs at a particular moment, but within a broad range of possibilities deemed useful by the master in step 1.

### Useful Settings may be Preprogrammed into the Invention for the Convenience of the User

As a convenience to the user, certain settings that have been determined to be useful (e.g. by a master) may be preprogrammed into the invention. For example, settings for particular chakras, acupuncture meridians, and others have been preprogrammed into the firmware of the embodiment of the present invention currently marketed as the PERKL-LIGHT. As such, if a user wants to experience energy of a vibration that is intended to balance a particular chakra, the user may put the invention on the setting for that particular chakra and spend time in proximity to the invention. These “preprogrammed settings” were referred to as “program modes” in the prior priority application.

### Useful Settings/Configurations may be Combined

One useful attribute of the present invention is that settings/configurations that have been determined useful may be combined. This could be done several different ways:

1. Using one instance of the invention running various settings in sequence. An example of this would be an instance of the present invention sequencing through preprogrammed settings for all of the chakras, in order to balance them. This is called a “program” (operational definition.) This functionality is described in prior priority applications and in this continuation in part application.

2. By operating multiple instances of the invention simultaneously. This can be used to increase the power (e.g. if the instances of the present invention are all on the same setting) or to provide an energy field of greater complexity, with energies with a plurality of different vibrations operating simultaneously.

3. Using combination of 1 and 2—i.e. more than one instance of the invention being used simultaneously, with one or more of the instances of the invention executing a program that sequences through a series settings/configurations that have been determined to be useful.

For example, a master may be involved with determining the best way to combine configurations and/or settings (including physical positioning and programs (operational definition). A master may be involved with determining programs (operational definition) for balancing chakras, acupuncture meridians, or for other purposes deemed useful by the master. Another example is that a master may be involved with determining configurations and/or settings and/or physical positioning of one or more examples of the present invention for the purpose of creating a sacred space. (See “Method for creating a sacred space” later in this description). A further example would be adding to this one or more examples of the present invention creating a sacred space, one or more examples of the present invention executing a program. For example, a master may determine settings/configurations/ (including physical positioning and programs (operational definition)) of examples of the present invention that are optimal for assisting one in balancing their chakras. The next step would be to add one or more examples of the present invention executing a program, such as sequencing through the seven chakras. In this way, a user exposed to the environment created thusly would have access to a powerful energetic experience engineered by the master. Indeed, this experience could be replicated—many such environments could be created for the benefit of users all around the world. All the environments created thusly would have energy with a consistent “vibration,” that was deemed useful by the master for a particular purpose, also deemed useful by the master.

### The Present Invention Provides a Platform for a New Art Form, in the Field of Chi or Subtle-Energy

Another way to think about the present invention is that it provides a platform for a whole new art form. As music is art in the medium of sound, and oil painting is art in the medium of visible light, the present invention provides a platform for a new art form in the field of chi or subtle energy. In the same way that an oil painter has an infinite way of combining the oil paints on a canvas, an artist of the present invention has an infinite number of configurations and/or settings (including physical positioning and programs (operational definition)) for the device, which produce different “vibrations” of subtle energy. Similarly to any art-form, some people will like a particular vibration, and others will not.

It should be recognized that while it is tempting to lump this invention in with art forms involving visible light (because some embodiments of the present inventions mix colors of light), this invention operates primarily in the medium of chi/life-force energy/subtle energy. This has been clearly shown because this invention is also capable of embodiments where all the light sources are the same color, and that therefore, do not mix colors of visible light. In this instance, it is easier to see that the differences in the energy are not due to perceived color of light, but rather, to differences in the setting/configuration of the invention.

Note that there are other art forms in the field of subtle-energy. People who work energetically with crystals will frequently choose them based on their subjective experience of the energy the crystal produces. There are many other examples of products that are chosen for the energy that they produce—merkabas, essential oils, sacred geometry products, chakra jewelry, etc. People who are sensitive to energy would generally agree that the Great Pyramids of Egypt are very well-known expressions of artwork in the realm of subtle energy.

With all of these, there is an element of artistry in the subtle energy that they produce. So in that regard, the notion of an art form in the area of subtle energy is not a new one. What is new is this particular technological platform for artistic expression in the field of subtle energy, made possible by the present invention. I.e. rather than, or in conjunction with crystals, sacred geometry products, chakra jewelry, etc., configurations and/or settings (including physical positioning and programs (operational definition)) of the present invention may be determined in order to make an energy that is deemed to have esthetic value.

An interesting point is that even though the science of subtle-energy is not yet well understood, it is possible to make a good embodiment of the present invention by thinking of it as an art form. The current state of the technology can be thought of as comparable to the state of wine-making in the 1700's. During this time, it was possible to make great wine, even though they did not yet understand the chemistry of what they were doing. Conversely, if you were to give a modern-day chemist the task of reverse-engineering wine, and then creating an imitation, you might not want to drink what he would make (unless, of course, the chemist was also skilled in the art of wine-making.) Similarly, an engineer who was not skilled in the energetic arts would be able to reverse-engineer the present invention, but it is unlikely that s/he would be able to make one with energy that people would like.

### The Present Invention has the Ability to Shift the Energy in Objects by “Clearing” them

In energy paradigms, it is commonly believed that all matter has chi/subtle energy associated with it. As described previously, a common notion in many energy paradigms is that people/places/objects can have a higher or a lower vibration. When a lower vibration is perceived, it is desirable to take steps to raise the vibration. “Clearing” is a word that is commonly used for the removal of negative (low-vibrational) energies, and their subsequent replacement with higher-vibrational energies.

An object can be cleared by prayer or blessing of the object. This prayer or blessing can also be thought of as the directing of chi toward the object. In this instance, the vibration of the person doing the blessing (or of the spiritual energies they access) can have a lot to do with how high the resulting vibration is of the object being cleared.

One highly useful capability of the present invention is its “clearing” mode, which is a means of raising the vibration of a person, place, or object. To clear an object, it is simply necessary to place the invention near the object and set it on its “clearing “mode. The clearing process may be enhanced by directing the energy from the invention toward the object being cleared by aiming the emitter toward it. It has been determined experientially that the energy radiating from the edges of a planar emitter are more forceful (a “yang” quality) than the energies one experiences when not in the plane of the emitter (which have a more “yin” quality). As such, these energies may be directed towards an object being cleared by holding/moving the invention so that the object passes through the plane of the (planar) emitter.

With other, (e.g. nonplanar) emitter configurations, a similar method may be determined consisting of:

1. Determining intuitively, the relationship to the emitter in physical space that is most effective for clearing an object

2. Placing the object being cleared in proximity to the emitter in the physical relationship determined in step 1.

In the embodiment of the invention currently marketed as the PERKL-LIGHT, the clearing mode is accomplished by adjusting the duty-cycle of a PWM signal applied to an LED that has been calibrated for optimal brightness and color, from 0% to 100% and back down again. A pleasant “rainbow” pattern is achieved by superimposing the brightness changes of two or more LEDs such that one is getting brighter while another is getting dimmer, while a third is off/resting. This particular embodiment of a clearing mode may be considered a “program” (operational definition), as it sequences very rapidly through a large number of configurations/settings.

This invention is capable of other embodiments for clearing. An aspect of the invention is a method in which a master determines optimal configuration(s), and settings, optionally including program(s), and/or physical positioning for optimal clearing. The intuitive gift of the master in making this determination is then replicated among a plurality of instances of the invention. As such, anyone who has possession of an instance of the invention also therefore, has access to the intuitive gifts of the master that have been replicated in the invention, and can clear using energies deemed optimal for this purpose by the master.

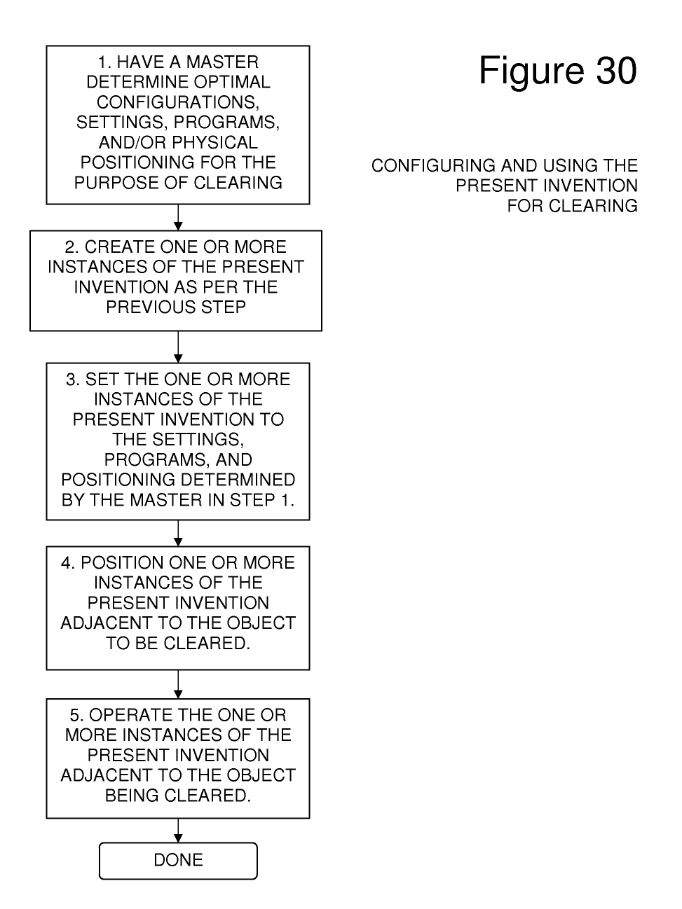

Here are the steps (see FIG. 30):

1. Have a master determine optimal configuration(s), and settings, optionally including program(s) (operational definition), and/or physical positioning of the present invention, for the purpose of clearing. Note that “configuration” includes the energetic definition of “programming.”

2. Create one or more instances of the present invention with the optimal configuration(s), settings, program(s), as per the previous step

3. Set the one or more instances of the present invention to the configuration(s), settings, program(s) (operational definition) and/or physical positioning determined by the master in the first step

4. Position these one or more instances of the present invention adjacent to the object to be cleared, optionally in a manner that has been determined to be optimal by the master (or that is determined intuitively to be optimal, by the people doing the clearing).

5. Operate these one or more instances of the present invention adjacent to the object to be cleared for a period of time determined by the master, or until the energy in the object is subjectively determined to have achieved a high vibration. Optionally, the present invention or the object may be moved relative to one-another, in order to direct the energy produced by the present invention toward the object being cleared at different angles.

### The Present Invention has the Ability to Create a “Sacred Space”

Similarly to clearing/raising the vibration of an object, a space such as a room or other physical location may be “cleared,” for the purpose of raising its vibration. A space that has been cleared in this manner is sometimes referred to as a “sacred space.” A sacred space contains chi/subtle energy that are of a high vibration. In addition to “clearing” the space of lower-vibrational energies (and then removing the instances of the present invention), a more powerful “sacred space” may be achieved through the continuous operation of one or more instances of the present invention with configuration(s), settings, optionally including physical positioning and/or programs deemed optimal by a master for this purpose.

It is desirable that a room in which an energy healer works on a person is of the highest possible vibration. In addition, it has been discovered that research experiments in the field of subtle-energy work better if they are conducted in a sacred space. Furthermore, it is desirable that any space that people inhabit be of the highest vibration, for the purpose of enhancing the experience of the people in that space.

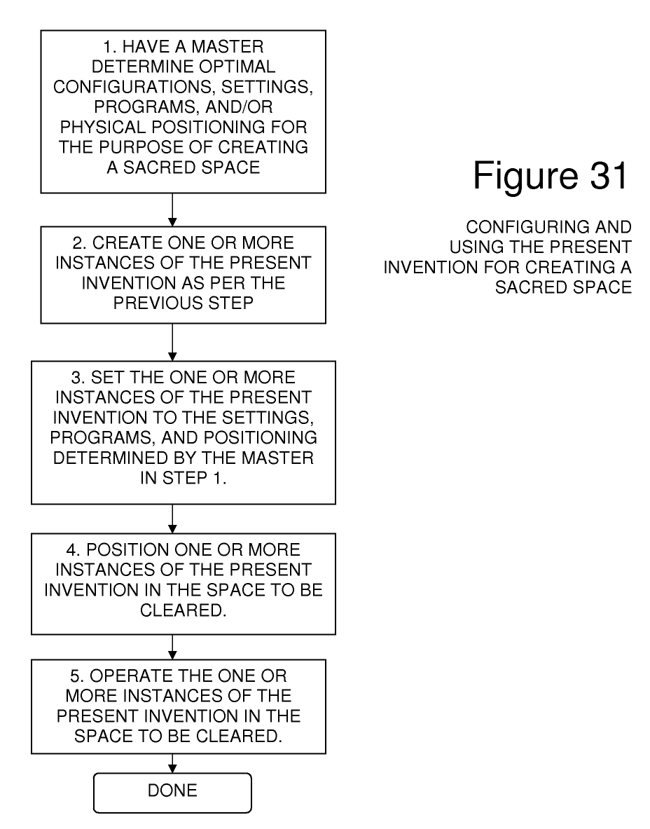

The present invention provides a new way of creating a sacred space. The method is as follows (see FIG. 31):

1. Have a master determine optimal configuration(s), settings, optionally including program(s) (operational definition), and/or physical positioning of the present invention, for the purpose of creating a sacred space. Note that “configuration” includes the energetic definition of “programming ”

2. Create one or more instances of the present invention with the optimal configuration(s), settings, and/or program(s) (operational definition), as per the previous step

3. Set the one or more instances of the present invention to the configuration(s), settings, program(s) (operational definition) determined by the master in the first step

4. Position these one or more instances of the present invention in the room or area to be cleared, optionally in a manner that has been determined to be optimal by the master (or that is determined intuitively to be optimal, by the people doing the clearing).

5. Operate these one or more instances of the present invention in the room or area for a period of time determined by the master, or until the energy in the room is subjectively determined to have achieved a high vibration. Optionally, the present invention may be moved around in the space being cleared, so that the energy from the present invention radiates from different angles relative to the space being cleared.

Note: Additionally, the “clearing mode” may be used prior to the steps above, to clear any negative (low-vibrational) energies.

An example of a method of clearing and creating a sacred space with the embodiment of the present invention currently being marketed as the “PERKL-LIGHT” is as follows:

1. Position one or more PERKL-LIGHT devices in the space to be cleared

2. Operate them on the “machine clearing” (a.k.a. “clearing) setting until it is determined intuitively that any negative energies have been removed

3. Set the PERKL-LIGHT device(s) on the “R-Space D-Space” setting (which is 29-red, 112-green, and 28 blue on an “Alpha Point” model of the PERKL-LIGHT).

4. Operate the PERKL-LIGHT device(s) in the space to be cleared until it has been determined that the energy in the space has a high vibration.

5. If needed, other settings may be determined intuitively, to clear and raise the vibration of the space.

### Methods for Working with the Biofield of in an Individual

Fundamentally, working on the biofield of an individual using the present invention is simply a matter operating the present invention in proximity to the individual being treated. As the biofield extends out beyond the body, it is not necessary for the user to remove their clothes.

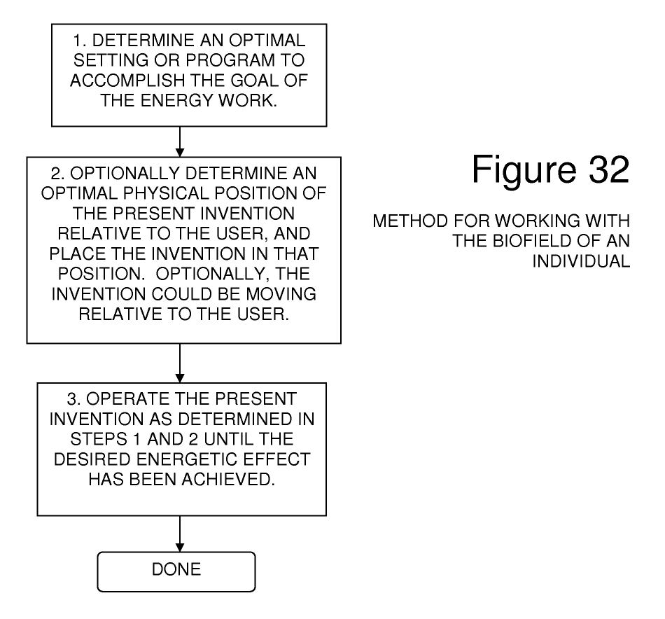

Here are the steps (see FIG. 32):

1. Determine an optimal setting or program to accomplish the goal for the energy work.

2. Optionally determine an optimal physical positioning of the present invention relative to the user, and place the present invention in that position. Optionally, the present invention could be moving relative to the user.

3. Operate the present invention as determined in steps 1 and 2 until the desired energetic effect has been achieved.

### Methods for Working with the Biofield of an Individual using Preprogrammed Settings

Preprogrammed settings provide a means of using the present invention that is very user-friendly. A user does not have to have any special intuitive gifts in order to use preprogrammed settings. If a master has been involved with determining the configurations that define the preprogrammed settings in a particular embodiment of the present invention, then all users of present inventions configured thusly will be able to take advantage of the intuitive gifts of the master, regardless of their own level of intuitive sensitivity.

Preprogrammed settings may be organized into “programs” (operational definition). This way, an embodiment of the present invention may sequence through a series of preprogrammed settings for a purpose determined to be useful by the master. For example, if the master determines preprogrammed settings for each chakra, these preprogrammed settings may be organized into a program (operational definition) such that the present invention will sequence from one chakra to the next, for the purpose of balancing them.

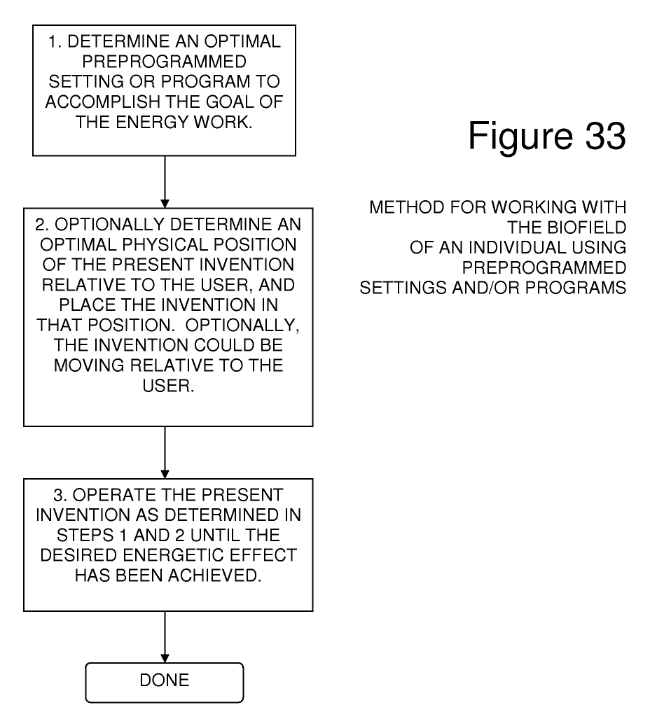

Here are the steps for working on the biofield of an individual using preprogrammed settings and/or programs (see FIG. 33):

1. Determine an optimal preprogrammed setting or program to accomplish the goal for the energy work. This may be determined by the user intuitively or by some other means, or by the master. For example, the master may determine a plan for working towards enlightenment that involves the use of one or more preprogrammed settings and/or programs (and/or manual settings, which are discussed in the next section of this description).

2. Optionally determine an optimal physical positioning of the present invention relative to the user, and place the present invention in that position (which could include moving the present invention relative to the user).

3. Operate the present invention as determined in steps 1 and 2 until the desired energetic effect has been achieved.

### Methods for Working with the Biofield of an Individual using Manual Mode Settings

While the preprogrammed settings are helpful, and can express the intuitive gifts of a master, it is also useful to have settings other than the preprogrammed settings available on a particular embodiment of the present invention. A manual mode allows one to manually adjust one or more user-adjustable configuration elements to create energy with vibrations not available in the preprogrammed settings. This allows for a great deal of flexibility and creativity in the use of the invention. For example, it is possible to find a setting that produces energy that engages a specific energy block that an individual has at a particular time. Further, it may be useful to find settings to create energy of a vibration that supports a particular intention, such as enhancing mental clarity, assisting one in furthering their progress toward enlightenment, or creating a sacred space.

To effectively determine appropriate manual mode settings, techniques are required for rapidly searching a vast space of possible settings. For example, on the embodiment of the present invention currently marketed as the PERKL-LIGHT, there are 16, 777, 216 possible manual mode settings (256 red settings X 256 green settings X 256 blue settings=16777216 possible combinations of red, green and blue). As stated previously, the use of the intuition and/or experience in the body makes it possible to search a vast problem space such as this rapidly. What follows are several techniques for accomplishing this:

### Scanning Technique

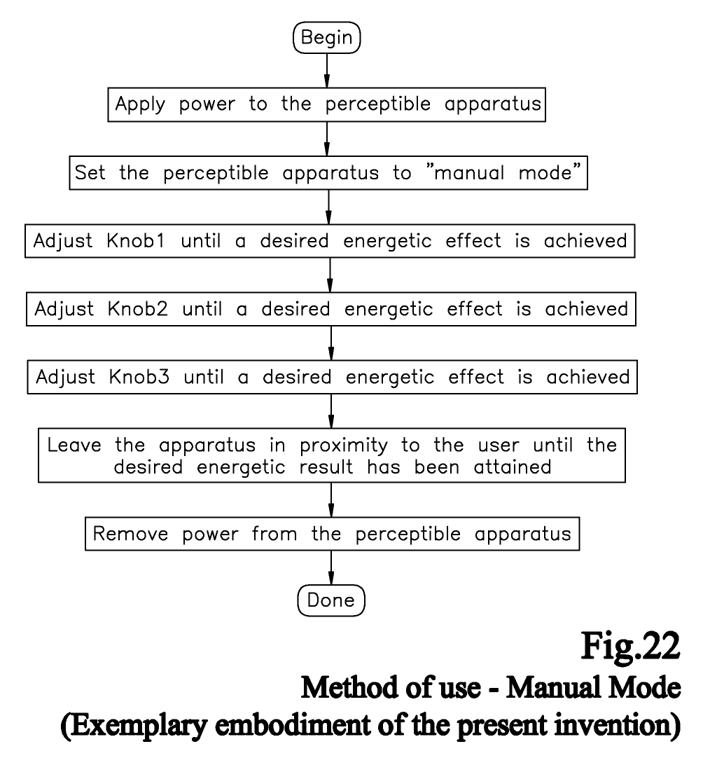

This material expands on a description presented in FIG. 22 of the prior priority application.

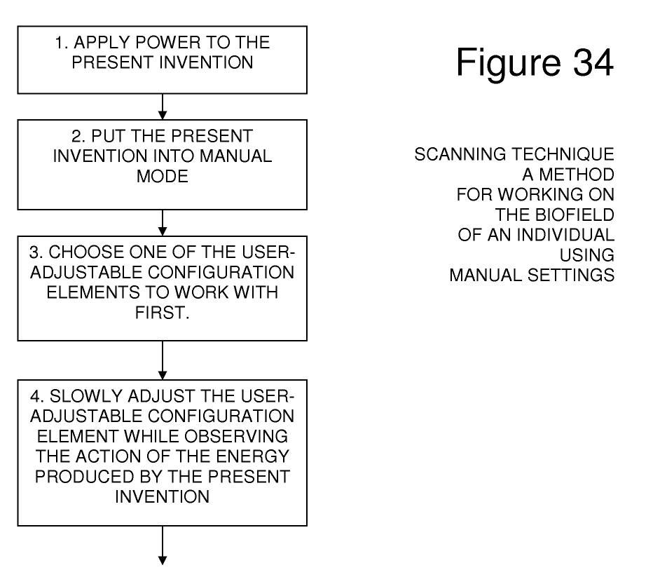

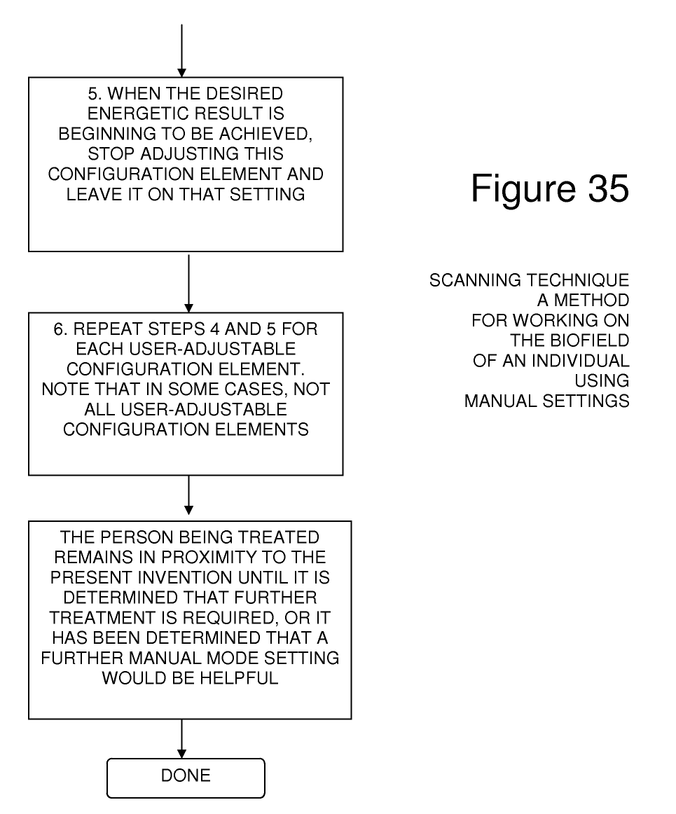

The scanning technique is a way that a person can find a manual mode setting on the present invention to create an energy field with a desired vibration, such as one to help clear an energy block. There are several variations on the scanning technique. The choice of which one to use depends somewhat on the skills and aptitudes of the user, on the type of energy work the user is engaging in (e.g. clearing an energy block in a user or creating a sacred space), and the nature of the condition the user is working on (e.g. is it a condition causing pain?). Here is the basic form of the scanning technique that is common to all the variations (see FIGS. 34 and 35):

1. Power up an example of the present invention

2. Put the present invention in manual mode

3. Choose one of the user-adjustable configuration elements to work with first

4. Slowly adjust this user-adjustable configuration element while observing the action of the energy produced by the present invention

5. When the desired energetic result is beginning to be achieved, stop adjusting this configuration element and leave it on that setting.

6. Repeat steps 4 and 5 for each user-adjustable configuration element. Note that in some cases, not all user-adjustable configuration elements will need to be used.

7. The person being treated remains in proximity to the present invention until it is determined that further treatment is not required (e.g. the pain has gone away or the energy block has cleared), or until it is determined that a further manual mode setting would be helpful.

The variations on the scanning technique all relate to steps 4 and 5—determining when the desired energetic effect is beginning to be achieved. Here are ways this can be done:

1. Experientially. This is the easiest to teach a person, as it requires no particular intuitive gifts or skill. This version of the scanning technique requires that the energy block you are working on is causing pain or discomfort or some other experience easily perceived by the senses of the body. While slowly adjusting a user-adjustable configuration element, the person monitors their level of pain/discomfort/other sensation. When the user notices even a slight or barely perceptible improvement in the pain/discomfort/other sensation, the user stops adjusting that particular user-adjustable configuration element, leaving it on that particular setting. The user might then repeat this process with any remaining user-adjustable configuration elements.

The inventor's interpretation of why this works in the case of pain is that pain is the body's way of signaling that something is wrong. Therefore, a slight improvement in the pain is a good indicator that something is happening that is improving the condition causing the pain. In this case, the slight improvement in the pain is indicating that the energy block is being cleared and that chi is flowing through the area in need of healing.

In our experience, this technique has proven highly effective at reducing or eliminating pain from a vast array of causes. It has also proven to be easy to teach to a person, regardless of their level of intuitive, energy-sensing ability.

2. Intuitively. This method for doing the scanning technique requires that the person using it has the ability to sense energy blocks in themselves or another person (whoever is receiving the treatment). Very simply, the person administering the treatment simply “observes” (intuitively) the energy block, and the energy produced by the present invention, while adjusting a user-adjustable configuration element. When the person giving the treatment observes that the energy produced by the present invention is engaging the energy block, they would stop adjusting that user-adjustable configuration element, leaving it on the setting determined thusly. They might repeat this process with any remaining user-adjustable configuration elements.

3. Using a testing modality. Many practitioners skilled in various forms of energy healing use a testing modality, such as muscle-testing, a pendulum, or the blink technique (which is taught on a DVD included with the embodiment of the present invention currently marketed as the PERKL-LIGHT.) Practitioners with these skills may use their testing modality to determine a manual mode setting by adjusting a user-adjustable configuration element while watching for a change in their testing modality. For example, if they are using a pendulum, they may slowly adjust the user-adjustable configuration element until the pendulum changes state (e.g. from “no” to “yes”). If they are muscle-testing, they may adjust the user-adjustable configuration element slowly while watching for the muscle-testing to change state (going from “strong” to “weak” works best). If they are using the blink technique, they may slowly adjust the user-adjustable configuration element until their eyelids twitch. Once they observe a change in their testing modality, they would stop adjusting this user-adjustable configuration element, leaving it on the setting determined thusly. They might repeat the process with any remaining user-adjustable configuration elements.

### Determining a Manual Setting Numerically

In addition to the scanning technique, a user may use a testing modality such as muscle-testing, pendulum, etc. to determine a manual setting numerically. The process is simple, but it does assume that the embodiment of the present invention has some sort of readout indicating the setting on a (one or more) user-adjustable configuration element(s).

To determine a setting numerically, the user simply divides the space of possible settings into subsets, and tests for which subset the best setting is in, using their testing modality. The process repeats recursively, until the desired answer is determined. This is similar to a binary search algorithm in computer science. For example, on the embodiment of the present invention currently marketed as the PERKL-LIGHT, the manual settings have a scale that runs from 0 to 255. To determine a manual setting numerically, the user would begin asking questions such as, “is the setting greater than 100?” If the user's testing modality gave a “yes” answer, they might then ask “greater than 200?” If no, “greater than 150?” If no, “greater than 125?” If yes, “greater than 135?” If yes, “greater than 140?” If no, “greater than 137?” If no, “is it 136?” If yes, then you are done, the setting for this user-adjustable configuration element is “136.”

### METHOD FOR INCREASING THE POWER OF THE PRESENT INVENTION

Use more than one of them at the same time. Particularly, it is possible to start more than one instance of the present invention simultaneously and increase the power. You can have them set on a setting that does not change, or have them all on a sequence such as chakras or acupuncture meridians, such that they sequence from one to the next in unison.

### DESCRIPTION OF THE PRESENT INVENTION

Before disclosing the exemplary embodiment of the perceptible apparatus 30 in detail, it is to be understood that the perceptible apparatus 30 is not limited in its application to the details of the exemplary embodiment disclosed, since the perceptible apparatus 30 is capable of other embodiments. Also, the terminology used herein as for the purpose of description and not of limitation.

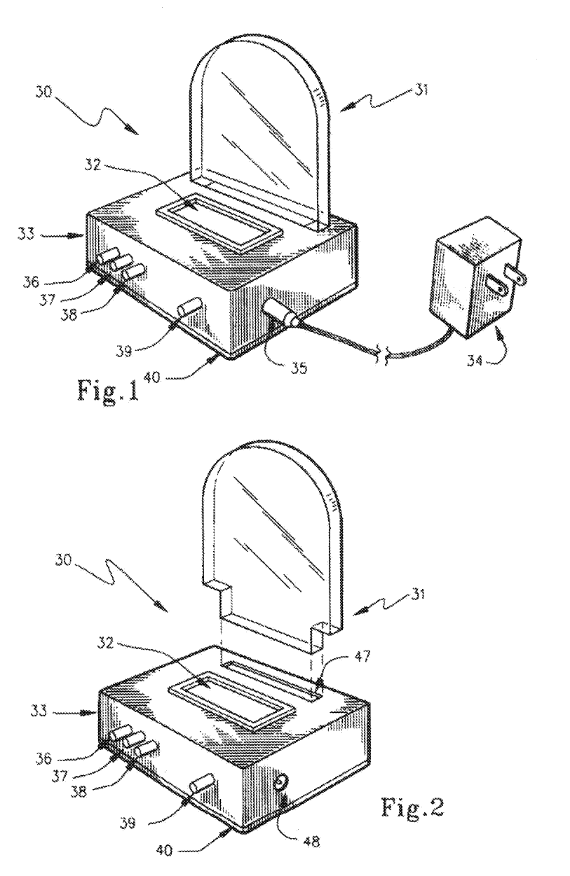

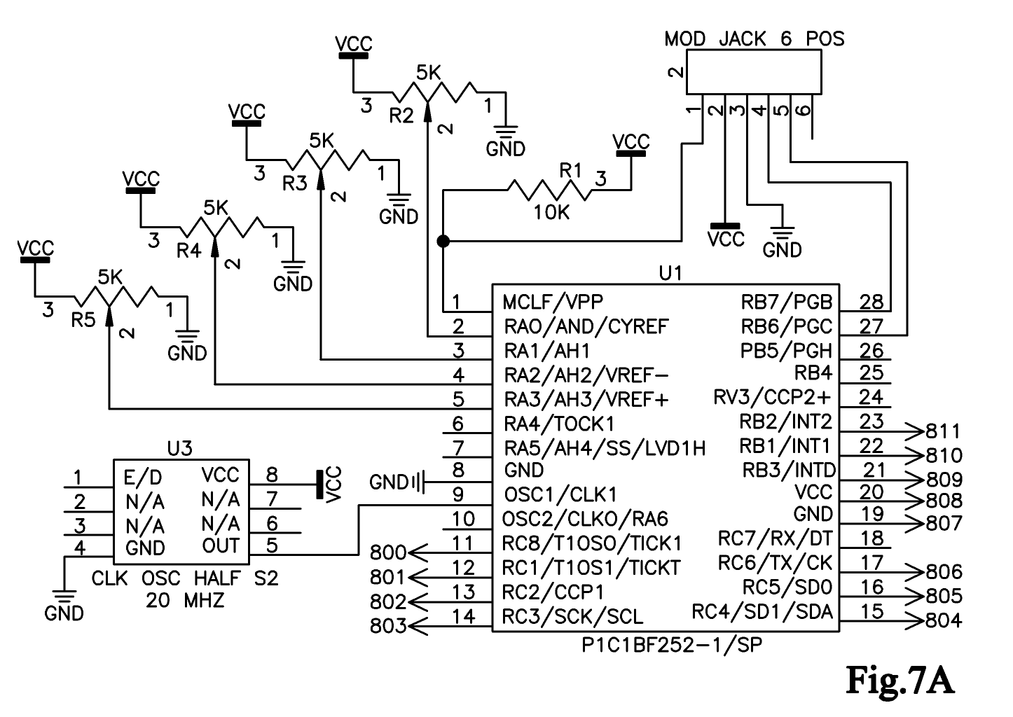

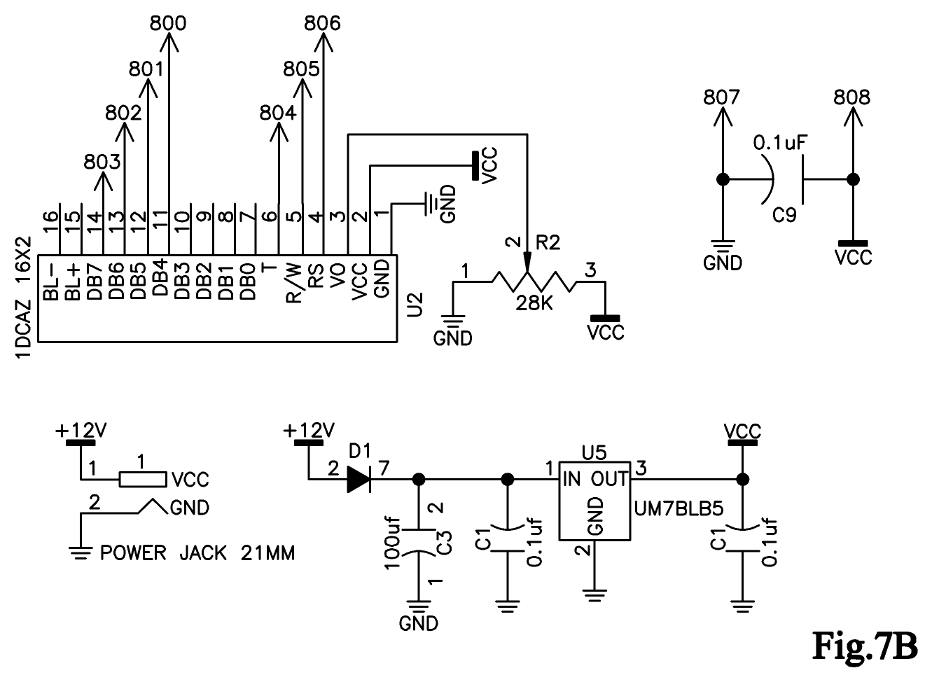

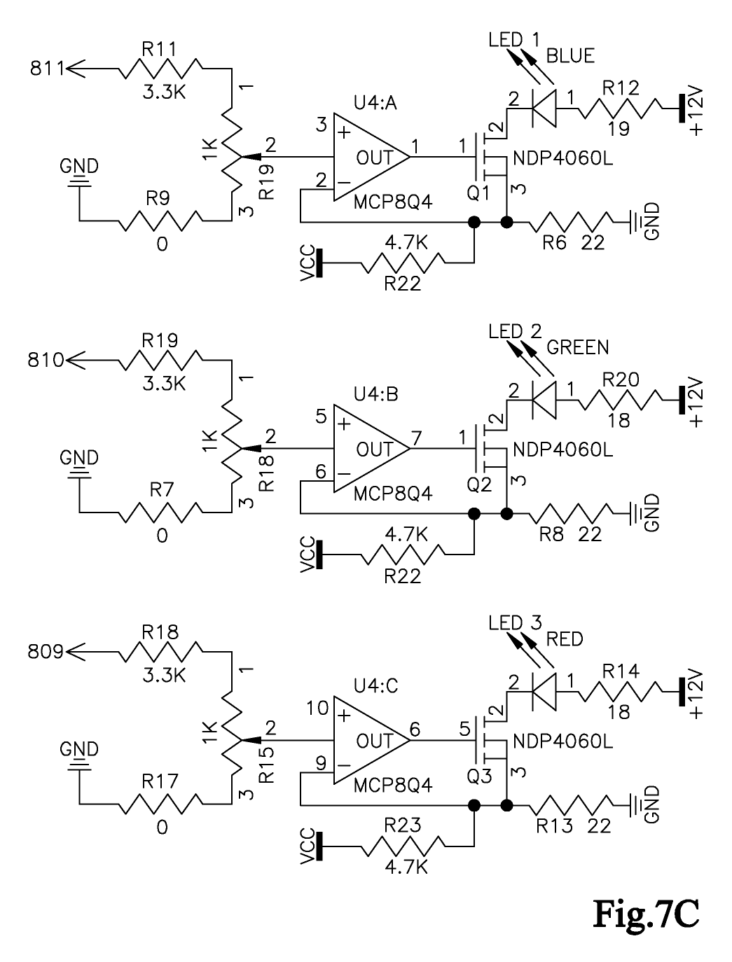

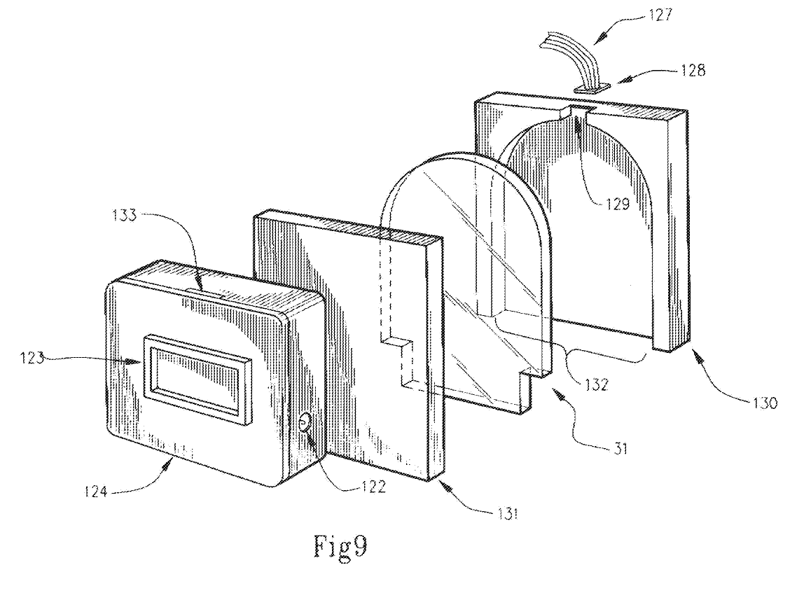

Initially referring to FIGS. 1-10, this invention operates by shining light at/through a subtle-energy producing material. The current exemplary embodiment of this invention uses red, green, and blue colored LEDs 44, 45, and 46 as the light source. These lights flash on and off at a rate that is too fast to be perceived visually (around 130 Hz in the current exemplary embodiment). The duty cycle of the individual lights is varied to produce the impression of the lights becoming brighter and dimmer. By adjusting the duty cycles of the red, green, and blue lights, it is possible to mix other colors of light. As red, green, and blue are the primary colors for light, virtually any color of visible light can be mixed using this scheme. The current exemplary embodiment of this invention has 256 different intensity levels for each color of light. As such, this device may generate over 16.7 million different colors.

It was discovered that polycarbonate has the property of being able to generate a subtle-energy field when light is directed towards and/or through it. The current exemplary embodiment of this invention uses red, green, and blue LEDs 44, 45, and 46, directed at the edge of a sheet of clear polycarbonate being the translucent element 31, to create a subtle-energy field. This embodiment has an LCD display 32 that shows the duty cycle that each color has been set to. Colors of polycarbonate other than transparent should be considered within the scope of this invention. Resolutions other than 8-bit (256 settings per channel) should be considered within the scope of this invention. The use of other kinds and colors of lights, including lights outside of the visible light field (e.g. infrared) should be considered within the scope of this invention. The use of other types of displays should be considered within the scope of this invention, although the use of displays should not be considered mandatory for the use of the present invention.

Lights, such as LEDs, are directed at or through a subtle energy-producing material, such as transparent or translucent polycarbonate in the form of a translucent element 31. The lights may be colored or white, or outside of the visible light spectrum (e.g. infrared). The lights may flash on and off, typically at a frequency that is too fast to be perceived. The lights may also flash from a brighter setting to a dimmer setting, rather than on and off. The waveform of the flashing lights may be square, sinusoidal, or some other form. The duty cycle may be varied. The intensity may be varied. The color of the lights themselves may be varied. The shape of the subtle energy-producing material may vary. Note that a display 32 can be optional, being not absolutely required for use of the present invention.

The present invention may be used in conjunction with biofeedback means, to determine an optimal setting for a particular result. The present invention may also have pre-programmed settings for a variety of purposes. The present invention would also include embodiments that are not adjustable at all.

This exemplary embodiment of the perceptible apparatus 30 comprises a circuit 51 in a housing 33, a translucent element 31 which protrudes from the housing 33, red, green, and blue LEDs 44, 45, 46 in the circuit 51 which direct light at the lower edge of the translucent element 31, an LCD display 32 that provides information about how the perceptible apparatus 30 is operating, adjustment knobs 36, 37, 38, and 39 which control the operation of the perceptible apparatus 30, and a wall-transformer 34 which provides power, and firmware which can be on a microcontroller U1 which controls the operation of this perceptible apparatus 30, thus the use of a microcontroller U1 and its associated firmware is optional, as other methods of providing a signal to the means 64 of producing the variable perceptible output should be considered within the scope of this invention.

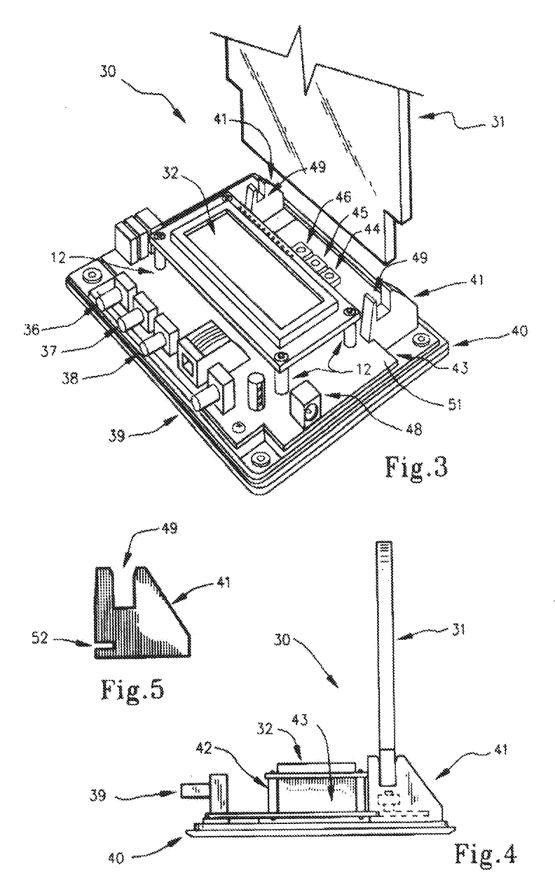

The exemplary embodiment of the perceptible apparatus 30 is for use in helping to create a reactive effect upon a user 54, and includes control circuitry 60 in conjunction with using optional microcontroller U1 and firmware that is operative to generate a one of a plurality of selected signals 62, which could preferably be pulse width modulation signals 62 at a substantially fixed frequency, or could be alternative types of signals 62. A means 64 for producing a variable perceptible output that is in response to one of the plurality of selected pulse width modulation signals 62. The means 64 is preferably the aforementioned LEDs 44, 45, and 46 of the colors red, green, and blue, however, the means 64 could simply be a singular or plurality of lights being preferably LEDs of any color combination or white alone, or anything that is perceptible or create a reactive effect in the user 54. Additionally, a translucent element 31 that is adjacent to the means 64 (as best shown in FIGS. 3, 4, and 6) is for producing a variable perceptible output and/or subtle energy, with the translucent element 31 being operative to diffuse and emit 65 the variable perceptible output/subtle energy. The translucent element 31 is preferably constructed of a synthetic thermoplastic resin and more particularly a polycarbonate, however, alternative materials that transmit a perceptible output or subtle energy would be acceptable. Also, optionally the perceptible apparatus 30 further includes a display 32 that is operational to indicate a relative level of the variable perceptible output.

Optionally, an adjacent reflective mirror 66 (as best shown in FIG. 21) can be added that is operational to further control the perceptible output by modifying the diffuse and emit 65 functions of the translucent element 31. Also, it is preferable that the perceptible apparatus control circuitry 60 are contained within a common housing 33 that is substantially parallelepiped in shape (as best shown in FIGS. 1 and 2) or other alternative shapes or configurations as desired for the common housing 33.

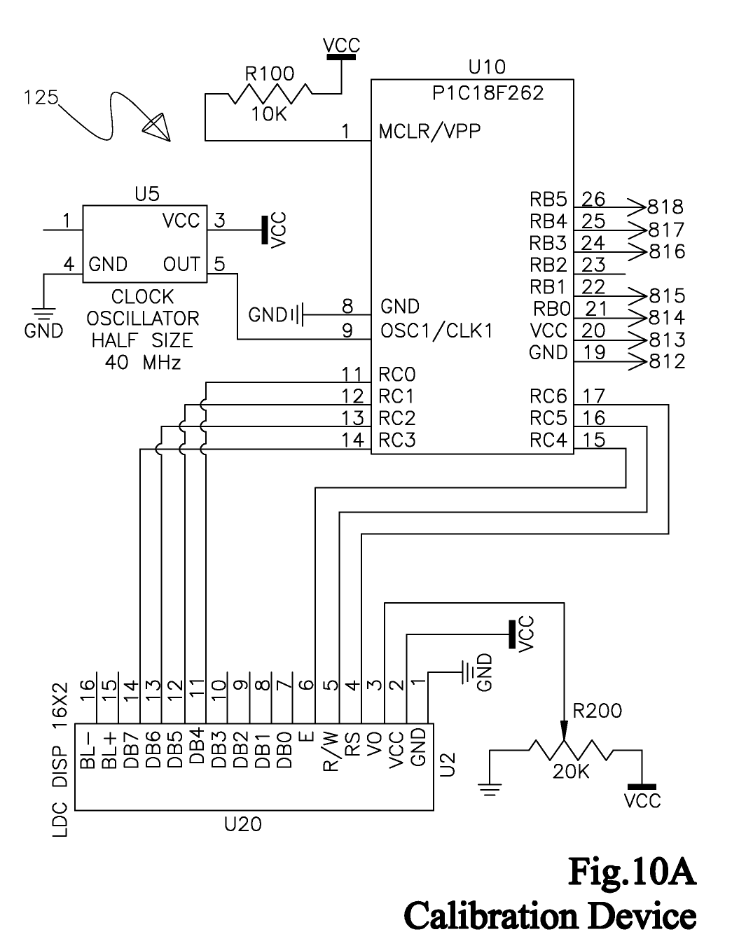

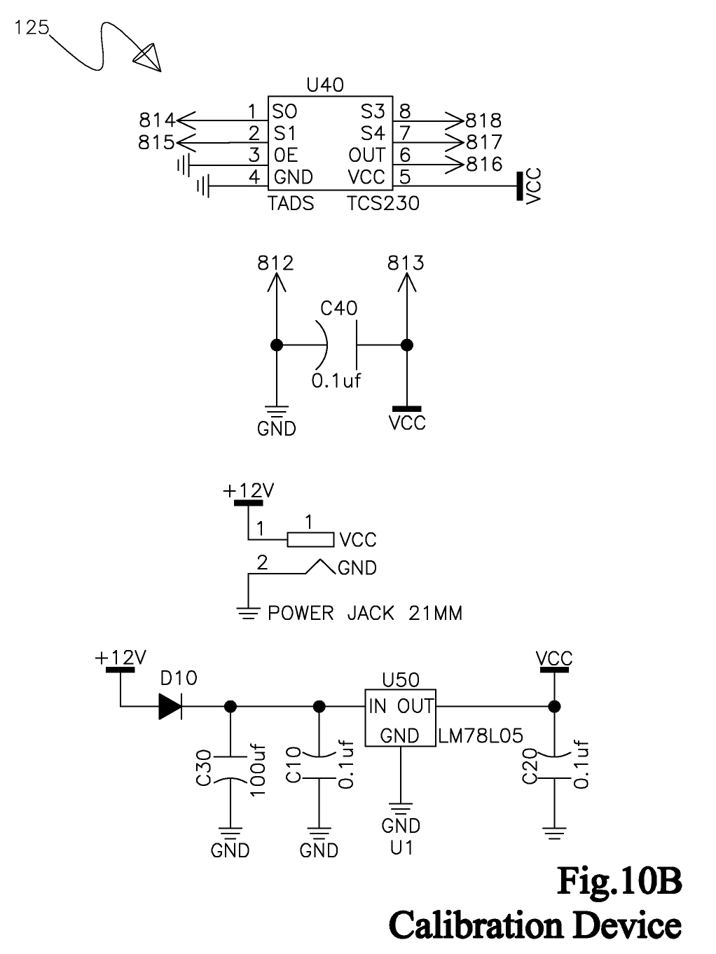

To further enhance or substantially standardize the subtle energy effect the perceptible output of the perceptible apparatus 30 the control circuitry 60 further includes calibration circuitry 68 for the LED of the means 64 for the variable perceptible output that is operational to help make brightness of the LED consistent amongst different LEDs from either different manufacturing batches or different units of the perceptible apparatus 30. To even further enhance or further substantially standardize the subtle energy effect the perceptible output of the perceptible apparatus 30 further includes a calibration device 121 that includes a shroud 126 that occludes substantially all external environment 134 light from the translucent element 31 and calibration device circuitry 125 (as best shown in FIG. 10) that provides a signal that is displayed in a display 123 indicating brightness and/or color of the LED. Brightness of the LED can be adjusted using the calibration device display 123 and the calibration circuitry 68 that is operational to further help make brightness of the LED consistent amongst different LEDs. Color of the LED can be adjusted by using the calibration device display 123 and replacing a selected LED that is operational to further help make color of the LED consistent amongst different LEDs.

As a further option, the perceptible apparatus 30 control circuitry 60 further includes clearing mode functionality selected by circuitry 70 in conjunction with firmware that upon activation is operational to replace the one of a plurality of selected pulse width modulation signals 62 with a continuously changing pulse width modulation signal 62 fluctuating between a maximum and minimum duty cycle with frequency remaining essentially constant.

The exemplary embodiment of the perceptible apparatus 30 for use in helping to create a reactive effect upon a user 54, includes control circuitry 60 operative to generate singularly or simultaneously in each one of a plurality of selected modes a plurality of selected signals 62 preferably being pulse width modulation signals 62 for each said mode. Also included is a plurality of lights being preferably LEDs 44, 45, and 46, with each one light variably illuminated in response to one of a plurality of selected signals 62 preferably being pulse width modulation signals 62 for each one of a plurality of selected modes. Further included is a translucent element 31 adjacent to the plurality of lights being preferably LEDs 44, 45, and 46, the translucent element 31 is operative to diffuse and emit 65 one light illuminated or a plurality of lights illuminated or for subtle energy. Optionally, the perceptible apparatus 30 for the translucent element 31 further includes an adjacent reflective mirror 66 (as best shown in FIG. 21) that is operational to further control the one light illuminated or a plurality of lights illuminated or for subtle energy. The translucent element 31 is preferably constructed of a synthetic thermoplastic resin and more particularly a polycarbonate, however, alternative materials that transmit a perceptible output would be acceptable that meet the aforementioned requirements. Also, it is preferable that the perceptible apparatus 30 control circuitry 60 be contained within a common housing 33 that is substantially parallelepiped in shape (as best shown in FIGS. 1 and 2), or other alternative shapes or configurations as desired for the common housing 33. Also, optionally the perceptible apparatus 30 further includes a display 32 that is operational to indicate a relative illumination level of each of the plurality of LEDs 44, 45, and 46.

To further enhance or for standardizing the energy effect of the perceptible output for the perceptible apparatus 30, the control circuitry 60 further includes calibration circuitry 68 for the LEDs of the means 64 for the variable perceptible output that is operational to help make brightness of the LEDs consistent amongst different LEDs from either the plurality of LEDs in a singular perceptible apparatus 30, different manufacturing batches of LEDs, or different units of the perceptible apparatus 30. To even further enhance or further standardize the energy effect the perceptible output of the perceptible apparatus 30 further includes is a calibration device 121 that includes a shroud 126 that occludes substantially all external environment 134 light from the translucent element 31 and calibration device circuitry 125 (as best shown in FIG. 10) that provides a signal that is displayed in a display 123 indicating brightness and/or color of the LEDs 44, 45, and 46. The brightness of the LEDs 44, 45, and 46 can be adjusted using the calibration circuitry 68 and the display 123 being operational to further help make brightness of the LEDs consistent amongst different LEDs. Color of the LEDs 44, 45, and 46 can be adjusted by using the calibration device display 123 and replacing a selected LED that is operational to further help make color of each one of the LED's consistent amongst the plurality of different LEDs.

It is possible, however, not mandatory to use LEDs that are of the same color, as an example an acceptable range for red is 622-645 NM (nano meters), with the dominant wavelength within 7 NM for blue and green having the dominant wavelength within 7 NM.

As a further option, the perceptible apparatus 30 control circuitry 60 further includes clearing mode functionality selectable by mode select circuitry 70 and/or firmware that upon activation is operational to replace said one of a plurality of selected pulse width modulation signals 62 for each selected mode with a continuous cycle of the red light 44 illuminated to a maximum sequencing to simultaneously reducing illumination of the red light 44 and increasing illumination of the green light 45 to a maximum illumination with the red light 44 not illuminated sequencing to simultaneously reducing illumination of the green light 45 and increasing illumination of the blue light 46 to a maximum illumination with the green light 45 not illuminated sequencing to simultaneously reducing illumination of the blue light 46 and increasing illumination of the red light 44 to a maximum illumination with the blue light 46 not illuminated.

### DESCRIPTION/OPERATION OF EXEMPLARY EMBODIMENT OF THE CALIBRATION CIRCUIT

An important goal of the exemplary embodiment of this invention (as well as many other possible embodiments) is that all perceptible apparatus 30 units will perform consistently with one another. This means that if a perceptible apparatus 30 is set to a particular setting, any other perceptible apparatus 30 could be set to that same setting, and the two perceptible apparatus 30 units would produce substantially identical subtle energy fields. Early in the process of developing this invention, it was believed that the color perceived by the eye was the important thing. As such, the obvious approach to calibration was simply to scale the duty cycles in software, to compensate for variations in the brightness of the LEDs. After some amount of development work, this idea was tested, and found not to work. Even though the colors were identical, the subtle energy fields they produced were very different. As such, it became necessary to devise another calibration scheme.

Through experimentation, it was determined that several factors matter in producing identical subtle energy fields. These factors are: the color of each LED, the brightness of each LED, the duty cycle of the LED, the frequency of pulsation of the LED, and the distance to the translucent element 31. The final three of these factors are easily controlled through engineering. Using a high-quality, bin-sorted LED controls the color of the LEDs. The brightness of the LEDs may be controlled by instituting current-regulating means into the circuit on the perceptible apparatus 30. Being able to calibrate for brightness allows for greater flexibility in selecting bin-sorted LEDs, possibly precluding availability problems when the perceptible apparatus 30 is in production.

After some experimentation, calibration circuitry 68 was developed, which allows adjustment of the current drawn by the LEDs when they are turned on, overcoming differences in brightness between different LEDs. This calibration circuitry 68 performs extremely accurate current sensing, so that use of an alternate power supply will not cause the perceptible apparatus 30 to consume a materially different amount of current. Thus, the perceptible apparatus 30 units will perform consistently, even if the power supplies vary substantially in voltage.

The following narrative describes the operation of the calibration circuit 68. For clarity, only one set of reference designators is (for the blue channel) used in this narrative. Operation is identical in the red and green channels as well.

Adjustment of the current drawn by the blue LED, being LED 46 is accomplished by controlling the voltage applied to the gate of power MOSFET Q1. The power MOSFET Q1 functions as an adjustable series-resistor, controlling the current through LED1. The calibration circuitry uses an op amp U4 to apply a voltage to the gate of the power MOSFET Q1. One input to the op amp U4 is the PWM signal from the PIC controller U1, scaled through a trimmer potentiometer R19. The other input to the op amp is a signal from a series-resistor R6, in series with LED1 46. This input creates a current-sense signal to the op amp U4. Calibration is accomplished by adjusting the trimmer potentiometer. The circuitry is such that the op amp U4 will try to make the voltages equal at its two inputs. It does this by changing its output, which is applied to the gate of the series MOSFET Q1, until the current-sense voltage is equal to the amplitude-scaled PWM signal from the PIC controller. In this way, the intensity of LED 46 during its on-setting may be controlled by adjusting the trimmer potentiometer R19.

One more nuance deals with the function of resistor R24. Without this resistor, the op-amp maintains enough voltage at the gate of the MOSFET Q1 to keep the LED 46 on slightly, when it is supposed to be off. R24 serves to artificially elevate the voltage of the current-sense input to the op amp U4, especially when the LED is in the “off” state. The result is that the op amp U4 will reduce its output to the gate of the MOSFET Q1 to essentially zero, causing the LED 46 to turn off completely. R24 may cause a slight change in the current flowing through LED 46 in the on state, but this is compensated for in the calibration process.

### DESCRIPTION/OPERATION OF EXEMPLARY EMBODIMENT OF THE CALIBRATION DEVICE

Referring to FIGS. 8-10, once it was known that the perceptible apparatus 30 units could be calibrated by controlling the color and/or adjusting the brightness of the LEDs during the “on” portion of their duty cycle, it was clear that a device was needed for measuring the brightness and/or color of the LEDs. Such a device was built as follows known as a calibration device 121.

A translucent element 31 was fabricated, identical to the translucent element 31 that are part of the perceptible apparatus 30. A black shroud 126 surrounds this translucent element 31, that occludes substantially all external environment light 134 from the translucent element 31. The only portion of the translucent element 31 that is un-shrouded is the part that protrudes into the translucent element slot 47 in the top of the perceptible apparatus 30. In this way, the only significant light that enters the translucent element 31 is the light from the LEDs 44, 45, and 46 themselves.

A slot 129 was cut in the top of the shroud 126, exposing a portion of the top edge of the translucent element 31. A TAOS TCS230 color-sensing chip 128 was placed in this slot 129, exposing it to the light coming from the translucent element 31. In this prototype embodiment, the TCS230 chip 128 was soldered via an adaptor to the ribbon cable 127. The TCS230 chip 128 was then immobilized in the slot with a blob of black RTV. The black RTV serves an additional purpose of sealing out room light that might otherwise enter the slot 129. An alternate design, using more than one TCS230 chip 128 and/or with the TCS230 chip(s) at different locations around the edge of the translucent element 31, should be considered within the scope of this invention. Using other means of mounting the TCS230 chip 128 should also be considered to be within the scope of this invention. Alternatively, other light and/or color measuring means, alternative to the TCS230 chip 128 could be utilized.

The TCS230 chip 128 is interfaced to the microcontroller U10. The TCS230 chip 128 outputs a pulse that increases in frequency, as the light gets brighter. The TCS230 chip 128 has an array of sensors, covered by red, green, blue, and clear filters. The TCS230 chip 128 can be programmed to output pulses corresponding to the brightness of red, green, blue, and the overall brightness of the light detected. In this way, the TCS230 chip gives an accurate measure of the color and/or brightness of the light striking its sensors. The microcontroller U10 simply displays on LCD display 123, the count of these pulses in a particular, arbitrary time period. The LCD display 123 gives four numbers—counts for red, green, and blue, as well as a count corresponding to the clear filter on the TCS230 chip 128. A circuit enclosure 124 containing the microcontroller U10, the LCD display 123, and related circuitry was attached to the shroud 126. The exemplary embodiment of the calibration device 121 is powered by a six-volt to nine-volt wall transformer similar to the one that powers the perceptible apparatus 30.

To use the exemplary embodiment of the calibration device 121, the translucent element 31 is removed from the perceptible apparatus 30 being calibrated. The calibration device 121 is inserted in place of the removed translucent element 31. Both devices are powered up. The calibration device 121 immediately begins displaying numbers, which correlate to the brightness of red, green, blue, and white, as detected by the TAOS chip 128. Choose a color, red, green, or blue, and turn its knob 36, 37, or 38 all the way up on the perceptible apparatus 30. The other two color knobs 36, 37, or 38 should be all the way down. The mode-select knob 39 should be in “manual” mode. The LEDs 44, 45, or 46 you have selected should be on steady, at 100 percent duty cycle. The calibration device 121 will display values for red, green, blue, and white. Adjust the trimmer potentiometer R15, R16, or R19, until the proper values are displayed on the calibration device. This adjustment needs to be done fairly quickly, as the LEDs 44, 45, or 46 gradually become less bright as they warm up. When the adjustment is complete, turn that LED's knob 36, 37, or 38 off, and repeat the process with each of the other two LEDs 44, 45, or 46. Once this is complete, the perceptible apparatus 30 should be calibrated. There is one final check that is advisable, however. This is to turn all three color knobs 36, 37, and 38 up all the way. This done, the calibration device 121 will show four numbers which indicate the color (hopefully white) that is produced with all three knobs turned up. This is a final “sanity check,” which is a little bit redundant, and therefore decreases the likelihood of an un-detected mistake in the calibration process.

It is also possible that if the LEDs within a batch are consistent enough, calibration could be accomplished by measuring the current drawn by the LEDs, rather than the light emitted. This is assuming that the proper current for this batch of LEDs had been predetermined using the calibration device. In this strategy, the “sanity check” described above (using the calibration device with all colors up full) would likely still be used, to make sure the LEDs are within spec.

The present invention is an apparatus 30 or a perceptible apparatus 30 for generating a subtle energy field 400 that includes a light source preferably in the form of LEDs 44, 45, and 46, or an equivalent, and a plurality of configuration elements 405 that are in effectual communication with the light source 44, 45, and 46, wherein the configuration elements 405 are adjustably selectable to produce subtle energy 400 of different vibrations 410. Note that effectual communication can be a combination of electrical communication, light wave communication, or any other medium that has a cause and effect relationship as between each of the configuration elements 405 and the subtle energy 400 both changing.

Further, the plurality of configuration elements 405 that are selected from the group consisting essentially of a spectral color analysis of said light source, a brightness of light pulses from said light source, a wave form of light pulses through a duration of light pulse(s), and a frequency of light pulse transitions from “on” to “off,” or transitions between different brightness levels. Also, the waveform is can based upon a square wave that can also optionally be pulse width modulation 415 (PWM). Wherein the pulse width modulation 415 (PWM) is sized and configured to be adjustable by changing the base frequency 420 of the PWM 415 waveform. Continuing, alternatively the pulse width modulation 415 (PWM) is sized and configured to be adjustable by changing the duty cycle of the PWM 415 waveform.

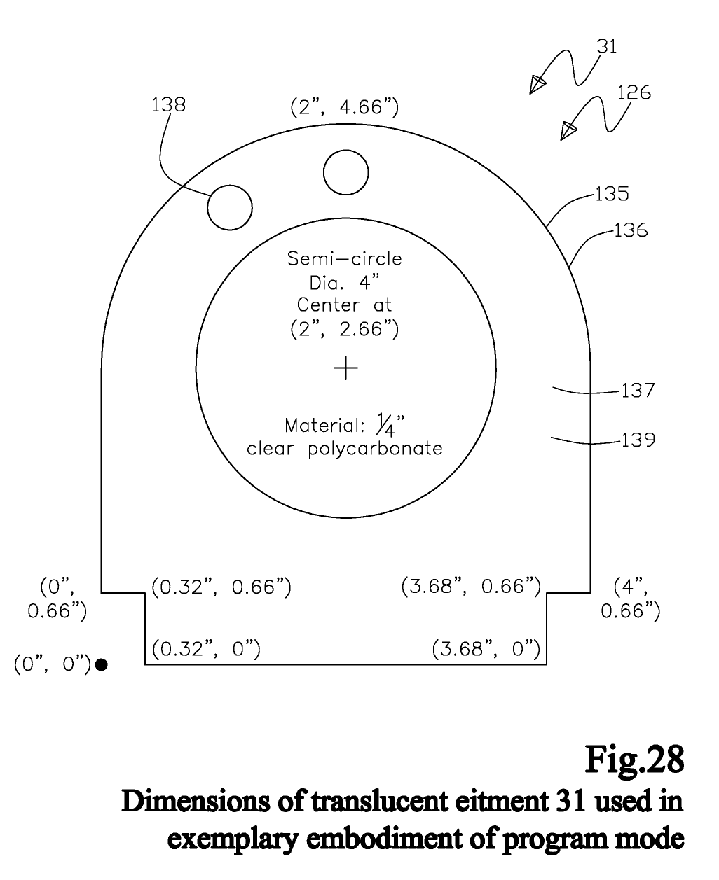

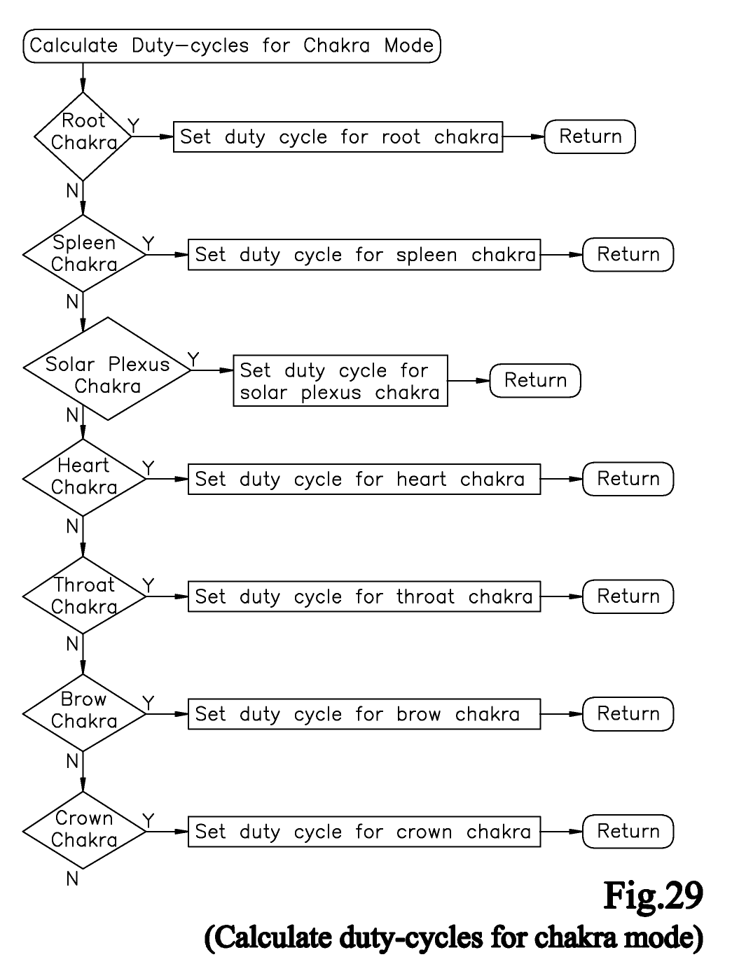

Also optionally, in looking at FIG. 28 in particular the configuration elements 405 can further include an emitter 425 in the form of a translucent element 31 that is in light wave communication with light source 44, 45, and 46 that is operational to modify the light source 44, 45, and 46 to produce changes in the subtle energy 400 field. Further, alternatively on the emitter 425 is constructed of a material that is selected from the group consisting essentially of a synthetic thermoplastic resin. Also, the emitter 425 can be constructed of a polycarbonate. In addition, optionally the emitter 425 can be constructed of quartz. Continuing, the emitter 425 has an annular shape 137 to effectuate a change in subtle energy 400. Next, the emitter 425 can optionally have a plurality of voids 138 disposed therethrough effectuate a change in subtle energy 400. Also, the emitter 425 can have a planar shape 139. Further, the emitter 425 has a periphery 135 with a textured surface 136 similar to a knurling or coarse surface texture.

### REFERENCE TO COMPUTER SOURCE CODE PROGRAM LISTING

This application includes a source code computer program termed firmware listing as an attached text file that was submitted in the claimed U.S. provisional patent application priority being U.S. provisional patent application Ser. No. 60/529,462 filed on Dec. 11, 2003 by David Thomas of Longmont, Colo., Bertrand Babinet of Lyons, Colo., and Jon Tempest of Parker, Colo., the contents of which are herein incorporated by reference.

### METHOD OF USE

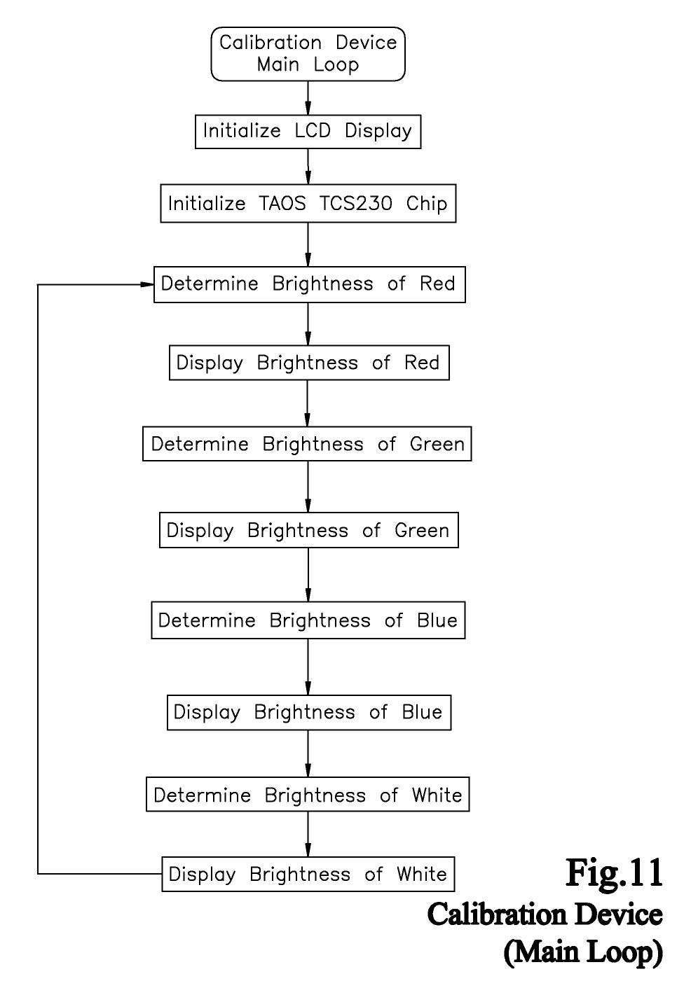

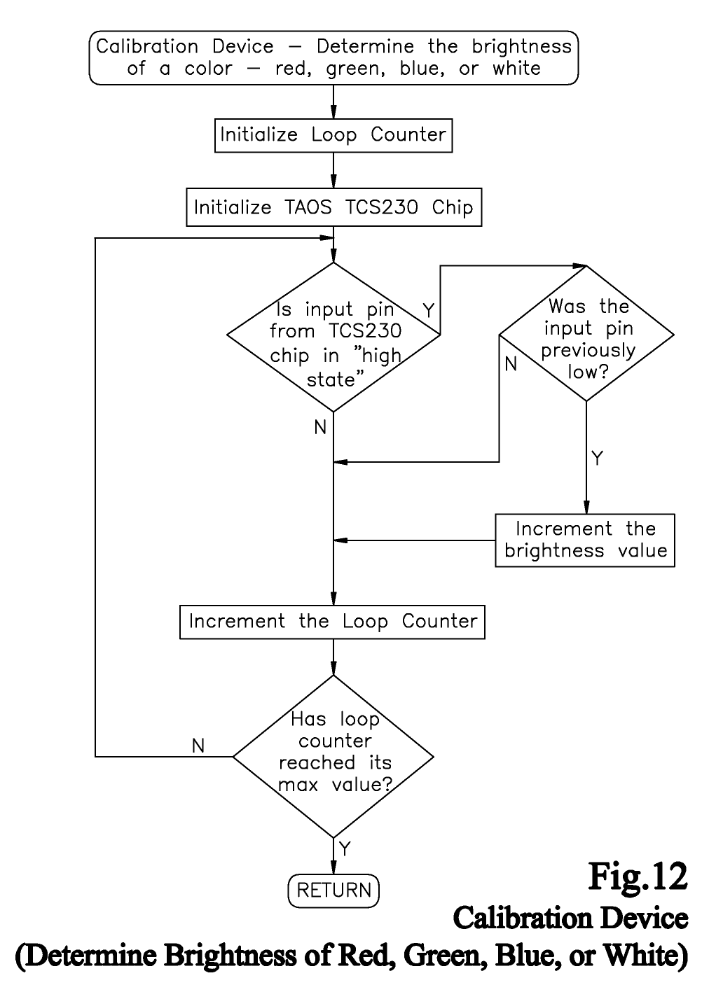

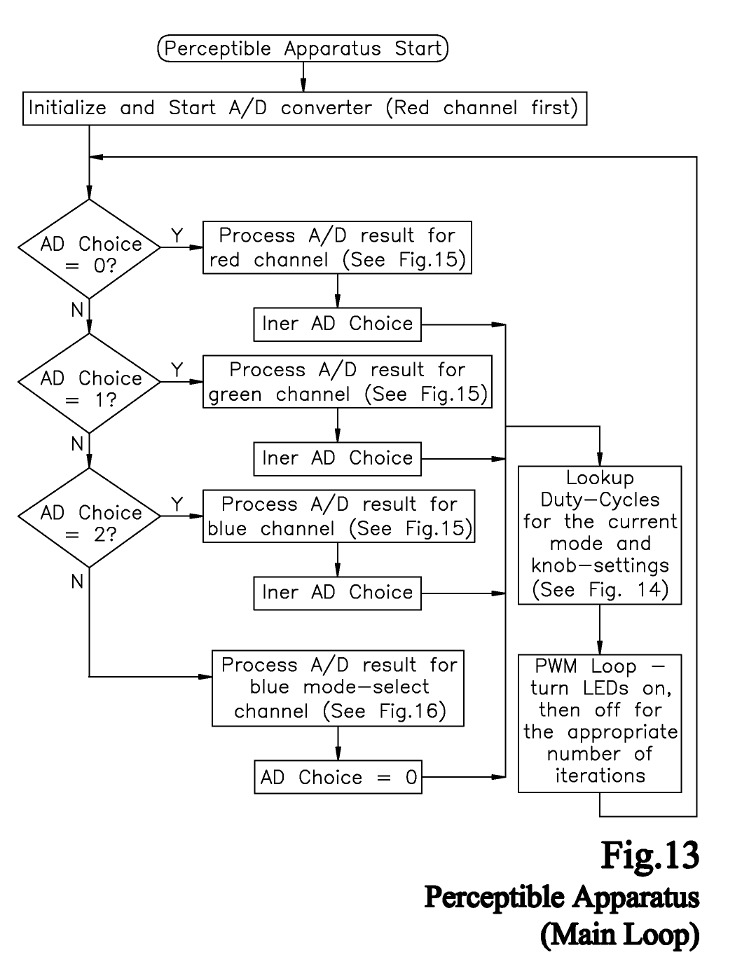

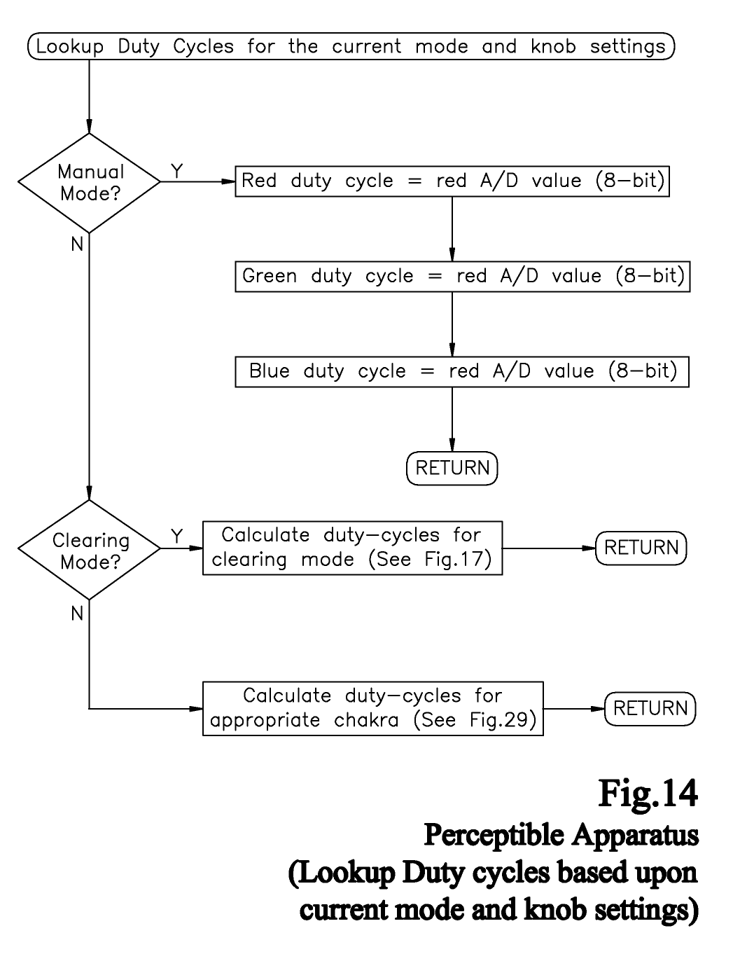

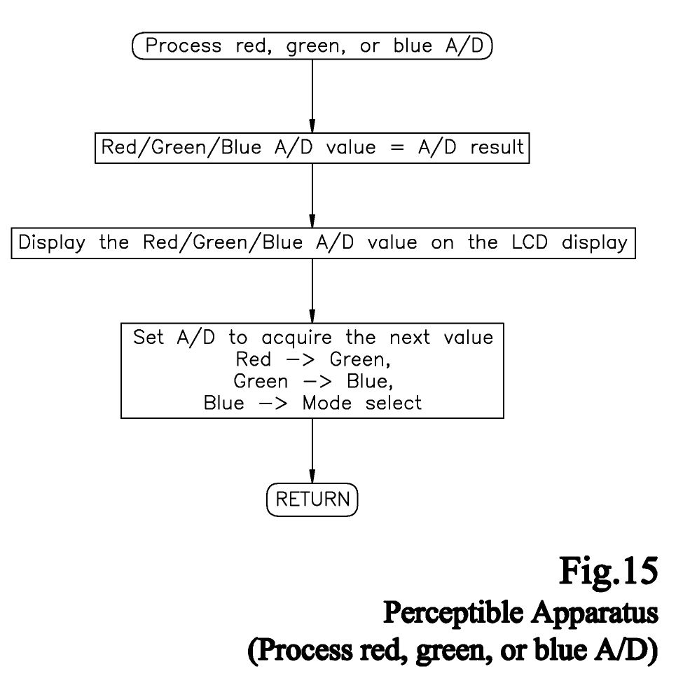

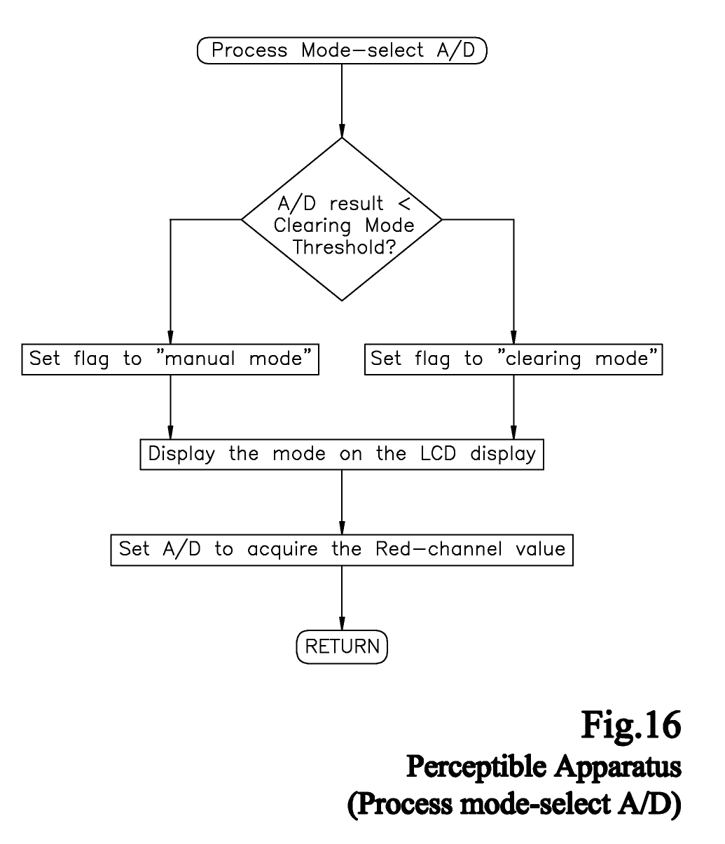

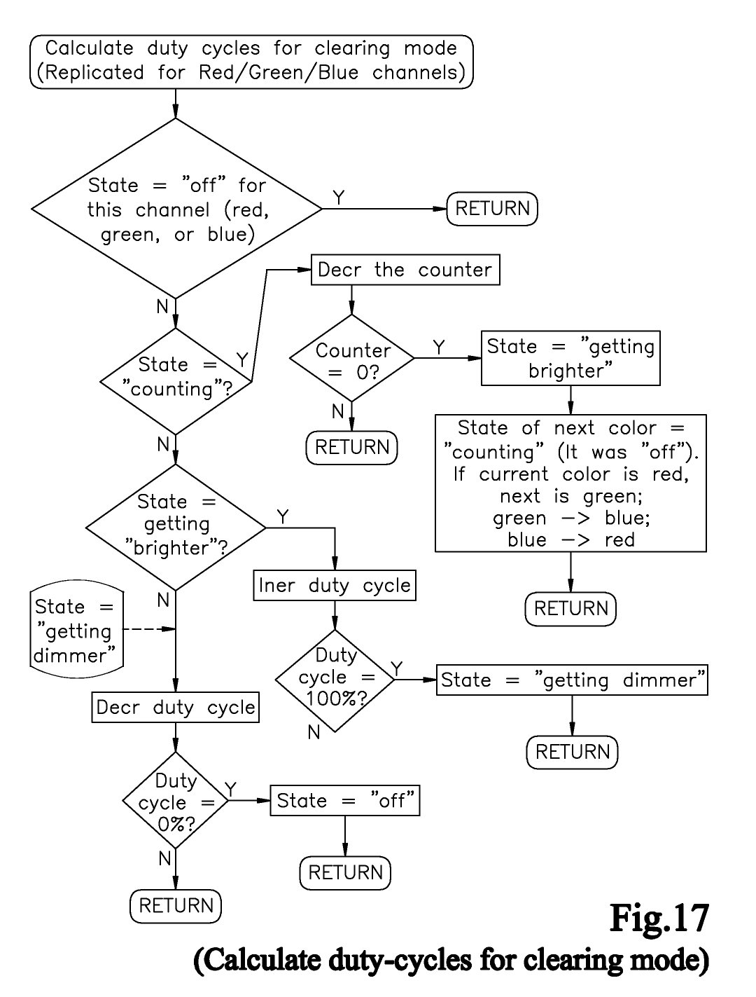

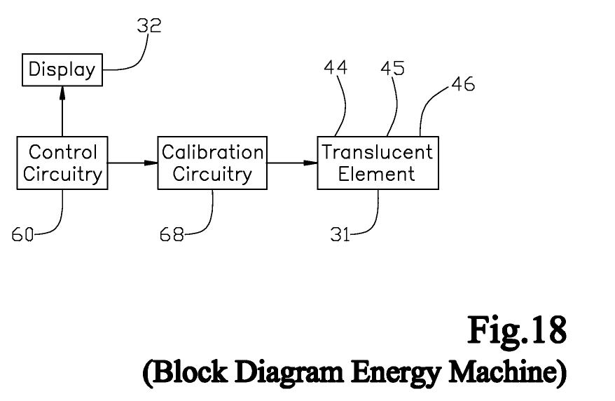

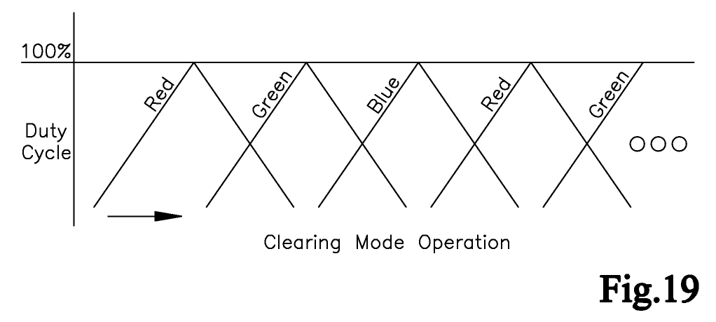

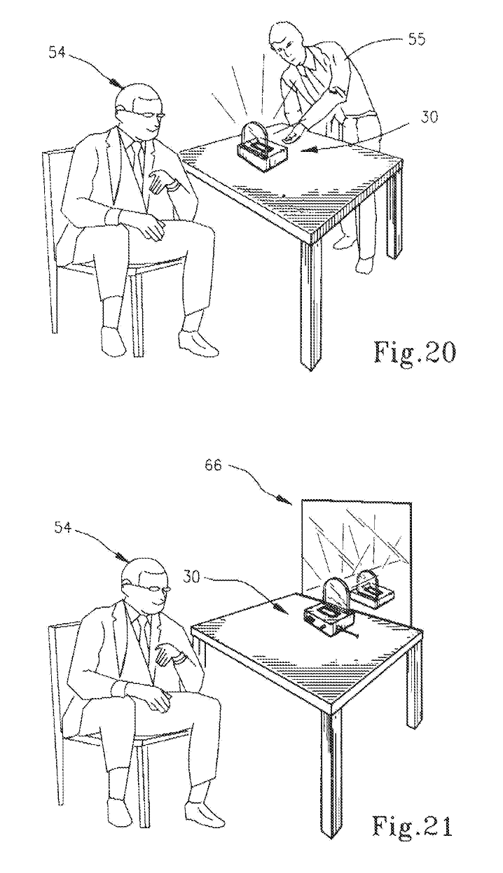

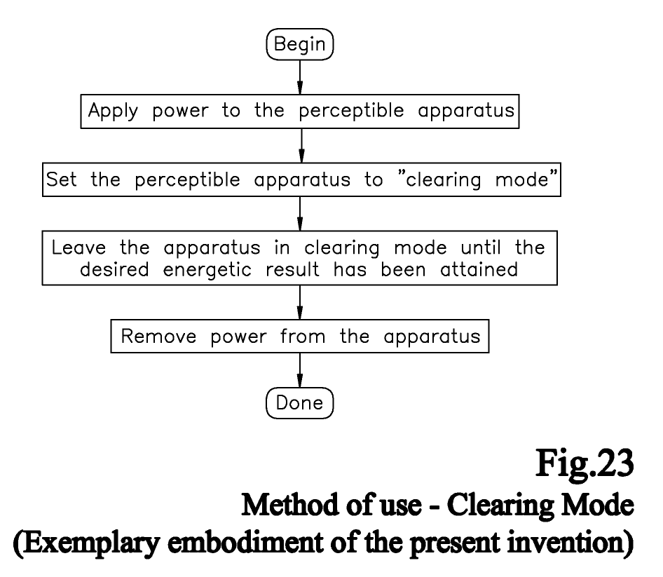

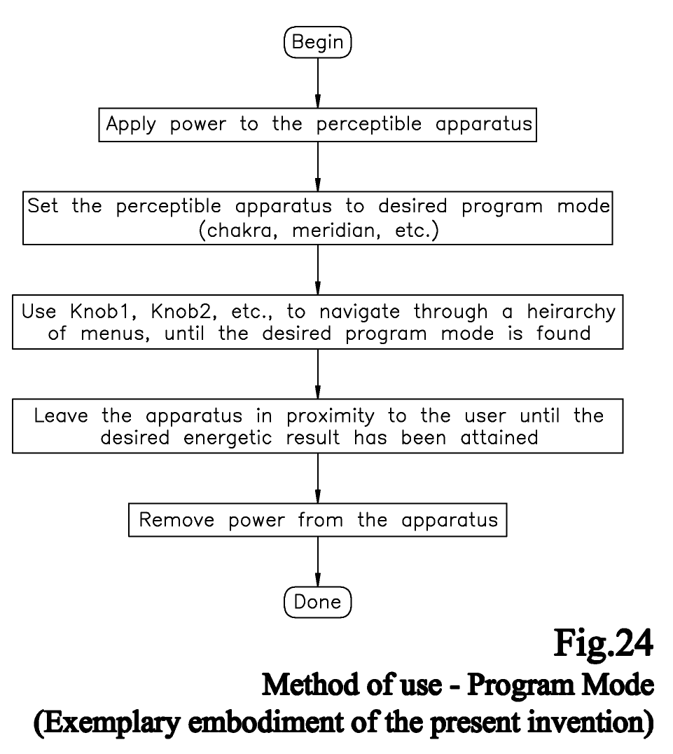

Referring to FIGS. 11-27, the exemplary embodiment of the perceptible apparatus 30 emits a perceptible output and/or subtle energy field, which is tunable, and which enhances and/or resonates with the biofield of the human body. Given the 8-bit resolution over three channels (red, blue, and green); over 16.7 million settings are possible. When the subtle energy field has been tuned to resonate with the energy field of the user 54, a useful/helpful result may be attainable. Tuning is accomplished by adjusting the duty-cycles of the three colors until a resonance occurs between the bioenergetic field of the user 54 and the subtle energy field produced by the perceptible apparatus 30. Rotating the three knobs 36, 37, and 38 makes this adjustment. The existence of this resonance may be determined by the intuition of the practitioner 55, or by the intuition/sensations experienced by the practitioner 55 or user 54. The practitioner 55 being defined as the administrator or controller of the perceptible apparatus 30, wherein the user 54 is seeking the desired reactive or energetic effect with possible help from the practitioner 55. In some cases, the user 54 fulfills the function of the practitioner 55. Alternatively, program modes may be incorporated into the perceptible apparatus 30, which may be used to generate a certain effect, such as balancing a chakra or acupuncture meridian. Additionally, means such as biofeedback may be incorporated, to determine an optimal setting for the perceptible apparatus 30. Either of these two alternatives makes the perceptible apparatus 30 more easily usable by the practitioner 55 or the user 54 who are less gifted intuitively.

### Manual Mode Operation

In the exemplary embodiment of the perceptible apparatus 30, manual mode operation is selected by rotating the “mode-select” knob 39 until the LCD display 32 indicates that you are in manual mode. In manual mode, the three other knobs 36, 37, and 38 take on the roles of adjusting the duty cycles of the red, green, and blue LEDs 44, 45, and 46. The color at the edge of the translucent element 31 changes as these knobs 36, 37, and 38 are adjusted. The display 32 shows a number between 0 and 255 for each color, indicating duty cycle or pulse width modulation signal 62. An intuitive practitioner 55 or user 54 would typically be necessary to set the perceptible apparatus 30 when operating in manual mode. Many practitioners 55 or users 54 are sensitive enough to set the perceptible apparatus 30 for themselves. Some practitioners 55 or users 54 are also sensitive enough to set the perceptible apparatus 30 for use with another practitioner 55 or user 54. A common way to set the perceptible apparatus 30 is to start with all the knobs 36, 37, and 38 turned off. Choose a knob 36, 37, or 38, and slowly turn it until a sense of energetic “resonance” is detected. Do the same with the other two knobs. It is not necessary for the user 54 to look at the perceptible apparatus 30 in order to receive treatment. Only the user 54 merely need to be in the vicinity of the perceptible apparatus 30, a couple of feet away works well for the user's 54 proximity to the perceptible apparatus 30. Sometimes, it works better to have the perceptible apparatus 30 in front of the user 54; at other times, it works better to have the perceptible apparatus 30 behind the user 54. At other times, it does not seem to matter where the perceptible apparatus 30 is.

A user 54 who is not intuitively gifted may use manual mode by having their practitioner determine the settings they should use, for example, at home, between appointments with the practitioner. The user 54 may then reproduce the settings at home by rotating the knobs 36, 37, and 38 until the proper values are displayed on the LCD display 32.

### Program Mode Operation

Program mode operation provides a way of setting the perceptible apparatus 30 appropriately, without requiring the presence of an intuitive practitioner 55 or user 54. Program modes are implemented by incorporating tables into the perceptible apparatus 30 containing predetermined settings. Program modes may be set up to support a variety of modalities. For example, you could have a program mode with table entries mapping to the chakras. A practitioner 55 or user 54 could then easily choose a setting to strengthen a particular chakra energetically. You could also have a program mode focused on acupuncture meridians. Program modes could be set up to energetically support the various organ systems of the body. Additionally, custom program modes could be set up to support many other healing modalities.

Program mode operation is selected by rotating the “mode-select” knob 39 until the desired mode of operation appears on the LCD display 32. In program mode operation, the other knobs 36, 37, and 38, rather than setting colors directly, are used to navigate through a hierarchy of menus to arrive at the desired “canned” selection. The selection may be a single color or it may be a sequence of colors. For example, a program could be created to cycle through the chakras, to balance them. It would also be possible to have a program mode for user-defined settings. This way, a practitioner 55 could program specific settings for a user 54, who could easily reproduce them at home. This might be more convenient than having the user 54 set the settings himself in manual mode.

### Biofeedback Mode Operation

Research needs to be done to determine appropriate biofeedback modalities that reliably indicate that the perceptible apparatus 30 has been properly set for a particular purpose. Early results using the Aurastar 2000 machine appear promising. Assuming such biofeedback modalities are found to be effective, it is strongly desirable to set up a closed—loop system comprising the biofeedback means and the perceptible apparatus 30. In one scenario, the biofeedback means would provide a signal to the perceptible apparatus 30. The perceptible apparatus 30 would try a variety of settings for each color, paying attention to the signal from the biofeedback means. The perceptible apparatus 30 would use this information to determine an optimal setting. In this way, no skill or special intuition would be necessary to use the perceptible apparatus 30 effectively. A simpler variation of this approach would be to have a human operator adjust the perceptible apparatus 30 while observing the output of the biofeedback means. Either approach should be considered to be within the scope of this disclosure.

It appears promising that the early results from testing of the Aurastar 2000 for biofeedback, wherein useful results were provided, thus, it seems likely that other biofeedback methods will be found that will work. This is because the perceptible apparatus 30 works extremely effectively in the hands of an intuitive practitioner. It is clear that the perceptible apparatus 30 is doing something in the body, because a wide variety of physical patterns of discomfort are often improved dramatically in a short period of time. This being the case, it seems very likely that some measurable indicator (probably more than one) will be found which indicates that the perceptible apparatus 30 has been set properly. (Examples are galvanic skin resistance (GSR), heart-rate variability, EEG, EMG, blood pressure, pulse rate, Kirilian photography, and other metrics known to those with skill in the art. Muscle testing has been found to be effective as a means of determining settings for the perceptible apparatus 30.) While the details of this mode of operation are yet to be fleshed out, the idea of using biofeedback as a means to properly set the perceptible apparatus 30 should be considered part of this invention.

### Clearing the Perceptible Apparatus 30

One of the challenges still present is a tendency of the perceptible apparatus 30 to occasionally pick up “stuff.” Often, the perceptible apparatus 30 seems to work by assisting the user 54 in releasing “negativity” from their body's energy field. Sometimes, the perceptible apparatus 30 seems to pick up this negativity, corrupting the subtle energy field produced by the perceptible apparatus 30 in subsequent sessions. The necessity of clearing the perceptible apparatus 30 seems to depend upon the severity of the energetic imbalance being corrected with the perceptible apparatus 30, with a particular user 54.

Sometimes this negativity can be removed from the perceptible apparatus 30 simply by cycling the perceptible apparatus 30 through the full range of red, green, and blue settings. This cycling has been automated by creating a clearing program, selected by the mode-select knob 39. In this clearing mode, the perceptible apparatus 30 fades from red, to green, to blue, and back to red, etc. continuously. The perceptible apparatus 30 cycles through all 1024 possible duty cycle settings for each color. The effect is that the perceptible apparatus 30 cycles through all the colors of the rainbow. One advantage of having an appealing clearing program is the user 54 will likely run the clearing program often, enjoying the pleasant effect it creates. This gives practitioner 55 or user 54 another way of enjoying the perceptible apparatus 30, but more importantly, encourages them to run the clearing mode a lot. It may be that many clearing mode algorithms will be found which work effectively, much as there are many screen-saver programs which work effectively on computers.

Another approach to clearing the perceptible apparatus 30 is to replace the translucent element 31 with a new one. A third approach is to expose the perceptible apparatus 30 to direct sunlight. The balanced, full-spectrum lighting of the sun seems to help clear negative resonance's picked up by the perceptible apparatus 30. A fourth approach to clearing the perceptible apparatus 30 is for a practitioner to use his/her intuition to determine a specific setting of the perceptible apparatus 30 that will clear the negativity. More research needs to be done to determine the best means of clearing the perceptible apparatus 30.

### Use of Clearing Mode as a Program Mode

Interestingly, in addition to clearing perceptible apparatus 30, the clearing program seems to have benefits with respect to balancing the user's 54 energy. As such, the clearing program may be used as a “program mode” as described above. The clearing program does not require any intuitive abilities to operate as a program mode. The clearing program has opened up a whole field of research to be explored, involving symmetrical, changing settings. It is believed that the symmetrical, changing settings are responsible for the clearing effect on the perceptible apparatus 30. Additionally, it is believed that these symmetrical, changing settings will produce useful results without requiring intuitive abilities on the part of the practitioner or user 54. This is because the symmetrical, changing settings cycle through the settings in a balanced way. The effect should be comparable to that of a tape-head demagnetizer, which neutralizes built-up magnetism in a tape head by exposing it to a balanced alternating-current magnetic field.

### Use of the Perceptible Apparatus 30 for Environmental Energetic Clearing

In addition to resonating with the biofield of a human, the perceptible apparatus 30 has potential applications for environmental energetic clearing. Practitioner 55 or user 54 who are sensitive to such things know that subtle energy fields are everywhere. There are a number of devices on the market that claim to emit a subtle energy field that balances harmful effects of subtle energy in the environment. Many of these devices are focused on strengthening a practitioner 55 or user 54 energetically so they are not bothered to the same degree by electromagnetic fields generated by electrical items in the home or workplace. The perceptible apparatus 30 appears to have application as an environmental clearing device. As of this writing, little experimenting has been done in this regard, however the clearing program described above seems to work effectively for environmental clearing.

### Use of Mirrors in Conjunction with the Perceptible Apparatus 30

As of this writing, a small amount of experimentation has been conducted in the use of mirrors 66 in conjunction with the perceptible apparatus 30. It has been observed that mirrors 66 have an effect on the subtle energy field produced by the perceptible apparatus 30. Placing the perceptible apparatus 30 in front of a large, flat mirror 66 tends to spread the energy field, rather than having it more concentrated around the perceptible apparatus 30. Experimentation needs to be done to determine the effects of other shapes of mirrors 66 on the subtle energy field. For example, would a parabolic mirror 66 create a focused beam of subtle energy, much like it does with light? Such an improvement might make it possible to focus a beam on a single user 54, or on a part of their body, without affecting others around them.

### Internal Operation of the Perceptible Apparatus 30

The exemplary embodiment of this invention has three LEDs, colored red 44, green 45, and blue 46. The LEDs 44, 45, and 46 flash at a rate too fast to be seen with the eye. Frequencies between 65 Hz and 260 Hz have been shown to work well. The duty cycle of the flashing LEDs 44, 45, and 46 varies between 0 and 100%, depending upon how the perceptible apparatus 30 has been set. The light produced by these LEDs is directed at the bottom edge of the translucent element 31. Black solder mask was used on the circuit board 43 to prevent the color of the circuit board 43 from influencing the color of the light being directed toward the translucent element 31. Additionally, the inside of the housing 33 is black. The bottom edge of the translucent element 31 is inside of the circuit housing 33. The top and most of the sides of the translucent element 31 are outside of the housing 33 and visible. In operation, the top and sides of the translucent element 31 illuminate with a color determined by the duty cycles of the red, green, and blue LEDs 44, 45, and 46. The perceptible apparatus 30 is controlled by a PIC 18F252 microcontroller U1, made by Microchip.

Four knobs 36, 37, 38, and 39 protrude through the front of the housing 33, and control the operation of the perceptible apparatus 30. These knobs 36, 37, 38, and 39 are part of potentiometers R2, R3, R4, and R5, the output of which is directed to four of the A/D converters in the PIC microcontroller U1. In manual mode, knobs 36, 37, and 38 control the duty cycles of the red, green, and blue LEDs 44, 45, and 46. The PIC microcontroller U1 reads the A/D converters attached to these potentiometers, and uses the resulting value to determine the duty cycle for the corresponding LED 44, 45, or 46. In a program mode, knobs 36, 37, and 38 are used to navigate through a series of menus appropriate to the program mode selected with the mode-select knob 39. This configuration provides a great deal of flexibility, as the user interface may be changed dramatically simply by upgrading the firmware, no hardware changes necessary.

A feature of the exemplary embodiment of this invention is the LCD display 32 on the top of the housing 33. This display 32 gives information about the operation (e.g. current settings) of the perceptible apparatus 30. Another feature is the calibration circuitry 68 incorporated into this embodiment of the invention. This calibration circuitry 68 enables the perceptible apparatus 30 to be calibrated, overcoming differences in brightness between different LEDs. This calibration circuitry 68 performs extremely accurate current sensing, so that use of an alternate power supply will not cause the perceptible apparatus 30 to consume a materially different amount of current.

### Method for Replicating a Desired Energetic Effect

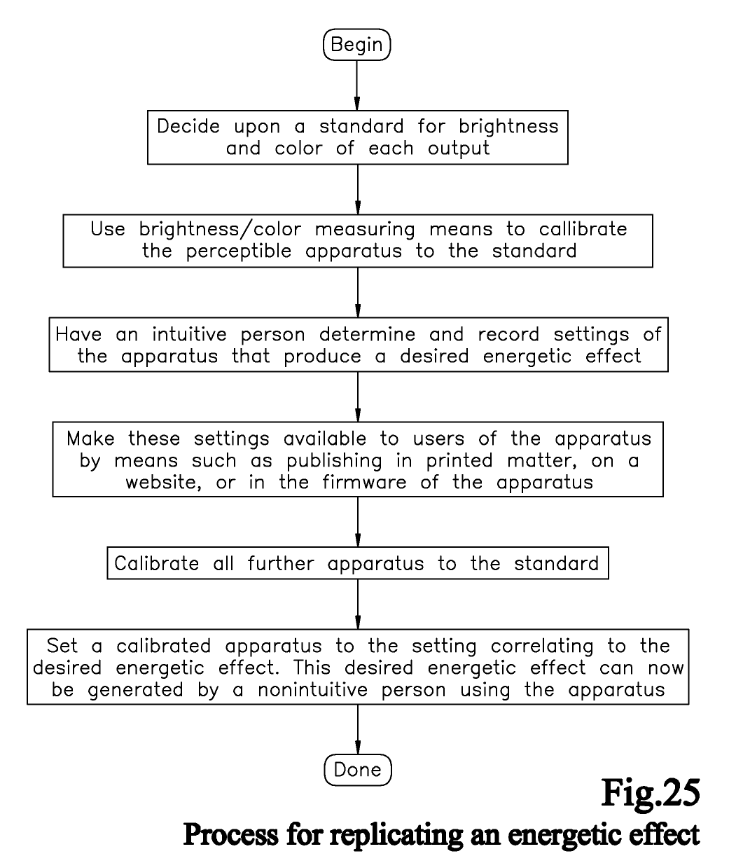

As stated elsewhere in this description, it is strongly desirable for the present invention to produce an energetic effect that is consistent from one perceptible apparatus 30 to the next for any given setting. This foundation of consistency is necessary to implement program modes. Fundamentally, the program modes are a means of replicating an energetic effect that has been deemed desirable. This section describes the overall method of replicating such desirable effects, and is described in FIG. 25.

The first step is to decide upon a calibration standard. The particular standard chosen appears to be somewhat arbitrary. The calibration standard will define the color and brightness of the light sources being LEDs 44, 45, and 46, in their on state. Consistency of color needs to be intrinsic to the light sources themselves in the exemplary embodiment of the perceptible apparatus 30. It has been found that an acceptable range of color is a dominant wavelength varying no more than 23 NM for red, 7 NM for green and 7 NM for blue.

Once the standard has been defined the perceptible apparatus 30 is calibrated to this standard, using color/brightness measuring means, such as the calibration device 121. This done, the settings necessary to produce desired effects are determined, for example through biofeedback means, or by an intuitive user 54. These setting would typically be used to support a particular modality, such as the chakra system, acupuncture meridians, and the like. These settings would then be made available to all users 54 of the perceptible apparatus 30 within that particular modality. They could then be made available either in program modes in the firmware in the perceptible apparatus 30, or published in some other way, such as printed matter or on a website. Once all this is done, it is possible for practitioner 55 or user 54 who are less intuitively gifted to use the perceptible apparatus 30 effectively to produce an energetic effect.

### Use of the Perceptible Apparatus 30 for Clearing a Crystal

There are a number of holistic modalities that use crystals. It is believed that certain crystals, such as quartz, have subtle energy with desirable qualities. Sometimes however, the subtle energy of a crystal can be corrupted through use in certain holistic modalities. It is possible to use the clearing mode on the perceptible apparatus 30 to clear the energy of the crystal. As shown in FIG. 27, remove the translucent element 31 from the perceptible apparatus 30 and then place the crystal 81 over the slot 47, such that the light from the LEDs 44, 45, and 46 shines through the crystal 81. Then put the perceptible apparatus 30 into the clearing mode, using mode select knob 39. The changing pattern of light generated by the clearing mode will neutralize the energy of the crystal, helping release negative energetic influences the crystal 81 may have acquired.

### Use of the Perceptible Apparatus 30 for Creating Essences

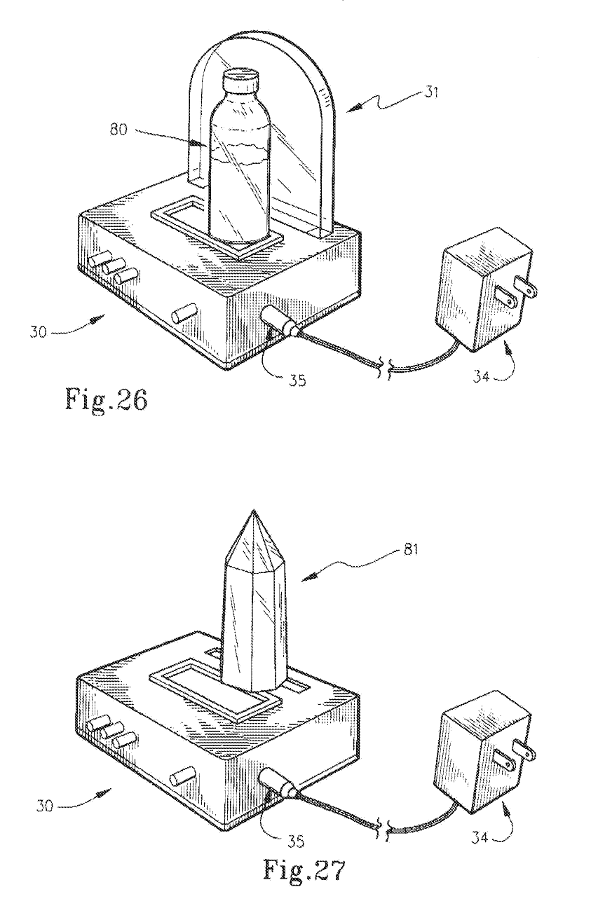

In addition to creating a subtle energy field that works directly on a user 54, perceptible apparatus 30 may be used to encode water or other liquids with a subtle energy. These “essences” that are created with the perceptible apparatus 30 are similar in some ways to homeopathic remedies. To create such an essence, simply set the perceptible apparatus 30 such that it produces a desired energetic effect, and then place a small container of water on top of the perceptible apparatus 30 as shown in FIG. 26 adjacent to the translucent element 31. After a period of time, being about 5 minutes or less, the liquid will take on the subtle energy produced by the perceptible apparatus 30.

### Method of using Balanced, Symmetrical Patterns of Operation for the Perceptible Apparatus 30

Defined as generic “energy balancing” program modes. These modes require no skill or special intuition on the part of the user 54 in using the perceptible apparatus 30. This can be used for energetically clearing the perceptible apparatus 30, a practitioner 55 or user 54 or animal, or an environment.

### PROGRAM MODE EXAMPLE

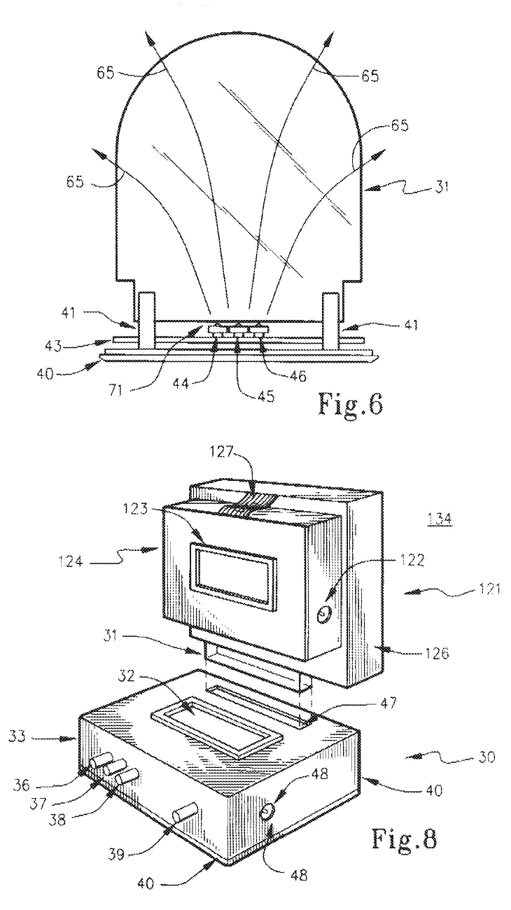

The information is this section is an example of a program mode reduced to practice. In order to make this an enabling disclosure, very specific examples, containing measurements, and the like are provided. This specific information is provided for the purpose of example, not limitation. With the measurements given a setting is provided which will resonate with the root chakra. The necessary measurements for the translucent element 31 in the perceptible apparatus 30 are given in FIG. 28. The measurements are the same for the translucent element 31 in the perceptible apparatus 30 and the translucent element 31 as used in the calibration device 121. In the calibration device 121, the TCS-230 chip 128 is centered at the top of the translucent element 31. The edges of the translucent element 31 are substantially textured, such that they illuminate with a color that represents a relatively uniform mixture of the light emitted by the LEDs 44, 45, and 46. The clearance 72 between the top of the circuit board 43 and the bottom of the translucent element 31 is about 0.28 inches, see FIGS. 5 and 6. The LEDs 44, 45, and 46 are centered under the bottom of the translucent element 31 in both the X and Y dimensions, as shown in FIG. 6. The LEDs are all “Superflux” LEDs made by Lumileds. The part No. of the red LED 44 is: HPWT-MD00, category code F04. The part No. for the green LED 45 is: HWPN-MG00, category code G17. The part No. for the blue LED 46 is: HWPN-MB00, category code B26.

The perceptible apparatus 30 is calibrated using the calibration device 121 to give the following brightness/color values: for the red LED 44; Red—9685, Green—217, Blue—101, and White—10, 293. For the green LED 45; Red—295, Green—3516, Blue—2578, White—10, 293. For the blue LED 46; Red—327, Green—1311, Blue—8454, White—10, 761. For consistency's sake, these measurements should be taken immediately after applying power to the LEDs 44, 45, and 46, as the numbers will tend to drop as the LEDs warm up.

In practice the perceptible apparatus 30 is calibrated by focusing on the number corresponding to the color of the LED being calibrated (i.e. to set the brightness of the red LED, adjust potentiometer R15 until the red number displayed 123 on the calibration device 121 is within +/−1% of 9685). The other numbers indicate the particular shade of red, and have an acceptable range of approximately +/−5%. Same process is used for the green LED 45 and the blue LED 46.

Once the perceptible apparatus 30 has been calibrated as described above, the following setting will resonate with the root chakra of a user 54: Red 235, Green 174, and Blue 243. The source code that is attached has a program mode for the root chakra implemented such that if the mode select knob is used to enter “chakra mode” the words “root” and “chakra” will appear on the display 32 and the perceptible apparatus 30 will operate in such a way as to resonate with the root chakra of a user 54.

If the measurements stated above are not followed diligently, the perceptible apparatus 30 will not resonate with the root chakra. However, other standards are possible, and may be equally effective. You would just need a different setting to resonate with the root chakra. With the input of an intuitive practitioner 55 or user 54, or through the use of machine such as the Aurastar 2000 (found to be effective at measuring the effects of the perceptible apparatus 30), other useful settings may be determined, and then replicated on all perceptible apparatus 30 units similarly calibrated.

### Methods of using and Calibrating the Perceptible Apparatus 30

The method of using the perceptible apparatus 30 for use in helping to create a reactive effect upon a user 54, includes the steps of first positioning the user 54, then providing the perceptible apparatus 30 that includes control circuitry 60 that is operative to generate a one of a plurality of selected signals 62, that are preferably pulse width modulation signals 62, with other signal types acceptable also. Also included in the perceptible apparatus 30 is a means 64 for producing a variable perceptible output in response to one of a plurality of the selected signals 62 and a translucent element 31 adjacent to the means 64 for producing a perceptible output, the translucent element 31 is operative to diffuse and emit the perceptible output. Further a next step is to locate the perceptible apparatus 30 to a selected position adjacent to the user 54. Subsequently, a step of activating the perceptible apparatus 30 being operational to illuminate the means 64 for producing the variable perceptible output that is in response to one of a plurality of the selected signals 62.

A further optional step is of deactivating the perceptible apparatus 30 and reactivating the perceptible apparatus 30 to re illuminate the means 64 for producing the variable perceptible output in response to another one of a plurality of different selected signals 62. Another optional step is of clearing the variable perceptible output 30 that is operational to continuously cycle a brightness of the means 64 for producing the variable perceptible output from a minimum setting to a maximum setting to a minimum setting.

A yet further optional step is to provide a perceptible apparatus 30 that includes control circuitry 60 operative to generate singularly or simultaneously in each one of a plurality of selected modes a plurality of selected signals 62 for each mode, a plurality of different colored lights, preferably being LEDs 44, 45, and 46 with each one light variably illuminated in response to one of a plurality of selected signals 62 for each one of a plurality of selected modes, and a translucent element 31 adjacent to the plurality of lights, the translucent element 31 is operative to diffuse and emit 65 one light illuminated or a plurality of lights illuminated, wherein the activating step initiates a selected sequence to illuminate each of the different colored lights to a selected illumination level.

Another yet further optional step 32 is a step of clearing, wherein the aforementioned different colored lights are operational to continuously cycle an illumination level of each different color light from a minimum setting to a maximum setting sequentially with the different colored lights.

Further a method of calibrating the perceptible apparatus 30 for use in helping to create a reactive effect upon a user, comprises the steps of providing a perceptible apparatus 30 that includes, control circuitry 60 operative to generate singularly or simultaneously in each one of a plurality of selected modes a plurality of selected signals 62 for each mode, a plurality of lights being preferably LEDs 44, 45, and 46, with each one LED variably illuminated in response to one of a plurality of selected signals 62 for each one of a plurality of selected modes, and a translucent element 31 adjacent to the plurality of the LEDs, the translucent element 31 is operative to diffuse and emit 65 one LED illuminated or a plurality of LEDs illuminated, further including calibration circuitry 68 for each one of the LED's that is operational to help make brightness of each one of the LED's consistent amongst the plurality of different LEDs. A further step is to provide a calibration device 121 that includes a shroud 126 that occludes substantially all external environment light 134 from the translucent element 31 and calibration device circuitry 125 that provides a signal that is displayed in a display 123 indicating brightness of the LEDs. Next a step of adjusting brightness using the calibration circuitry 68 and the calibration device display 123 that is operational to further help make brightness of each one of the LED's consistent amongst the plurality of different LEDs.

Yet further a method of calibrating the perceptible apparatus 30 for use in helping to create a reactive effect upon a user, comprises the steps of providing a perceptible apparatus 30 that includes, control circuitry 60 operative to generate singularly or simultaneously in each one of a plurality of selected modes a plurality of selected signals 62 for each mode, a plurality of lights being preferably LEDs 44, 45, and 46, with each one LED variably illuminated in response to one of a plurality of selected signals 62 for each one of a plurality of selected modes, and a translucent element 31 adjacent to the plurality of the LEDs, the translucent element 31 is operative to diffuse and emit 65 one LED illuminated or a plurality of LEDs illuminated, further including calibration circuitry 68 for each one of the LED's that is operational to help make brightness of each one of the LED's consistent amongst the plurality of different LEDs. Subsequently a step of providing a calibration device 121 that includes a shroud 126 that occludes substantially all external environment light 134 from the translucent element 31 and calibration device circuitry 125 that provides a signal that is displayed in a display 123 indicating brightness and color of the LEDs. A next step of adjusting color by using the calibration device 121 and the calibration device display 123 by replacing a selected LED that is operational to further help make color of each one of the LED's consistent amongst the plurality of different LEDs.

### CONCLUSION

Accordingly, the present invention of a perceptible apparatus has been described with some degree of particularity directed to the embodiment(s) of the present invention. It should be appreciated, though; that the present invention is defined by the following claims construed in light of the prior art so modifications or changes may be made to the exemplary embodiment(s) of the present invention without departing from the inventive concepts contained therein.

1. An apparatus for generating a subtle energy field, comprising:
(a) a light source; and
(b) a plurality of configuration elements that are in effectual communication with said light source, wherein said configuration elements are adjustably selectable to produce subtle energy of different vibrations.

2. An apparatus for generating a subtle energy field according to claim 1 wherein said plurality of configuration elements that are selected from the group consisting essentially of a spectral color analysis of said light source, a brightness of light pulses from said light source, a wave form of light pulses through a duration of light pulse(s), and a frequency of light pulse transitions from “on” to “off,” or transitions between different brightness levels.

3. An apparatus for generating a subtle energy field according to claim 2, wherein said waveform is based upon a square wave.

4. An apparatus for generating a subtle energy field according to claim 3, wherein said square wave waveform is pulse width modulation (PWM).

5. An apparatus for generating a subtle energy field according to claim 4, wherein said pulse width modulation (PWM) is sized and configured to be adjustable by changing the base frequency of the PWM waveform.

6. An apparatus for generating a subtle energy field according to claim 4, wherein said pulse width modulation (PWM) is sized and configured to be adjustable by changing the duty cycle of the PWM waveform.

7. An apparatus for generating a subtle energy field according to claim 2, wherein said configuration elements further include an emitter in the form of a translucent element that is in light wave communication with said light source that is operational to modify said light source to produce changes in the subtle energy field.

8. An apparatus for generating a subtle energy field according to claim 7, wherein said emitter is constructed of a material that is selected from the group consisting essentially of a synthetic thermoplastic resin.

9. An apparatus for generating a subtle energy field according to claim 8, wherein said emitter is constructed of polycarbonate.

10. An apparatus for generating a subtle energy field according to claim 7, wherein said emitter is constructed of quartz.

11. An apparatus for generating a subtle energy field according to claim 7, wherein said emitter has an annular shape to effectuate a change in subtle energy.

12. An apparatus for generating a subtle energy field according to claim 7, wherein said emitter has a plurality of voids disposed therethrough effectuate a change in subtle energy.

13. An apparatus for generating a subtle energy field according to claim 7, wherein said emitter has a planar shape.

14. An apparatus for generating a subtle energy field according to claim 7, wherein said emitter has a periphery with a textured surface.
